(19) United States
(12) Patent Application Publication
(10) Pub. No.: US 2010/0178355 A1
(43) Pub. Date: Jul. 15, 2010

(54) METHOD FOR IN SITU SOLIDIFICATION OF BLOOD-POLYMER COMPOSITIONS FOR REGENERATIVE MEDICINE AND CARTILAGE REPAIR APPLICATIONS

(76) Inventors: Caroline D. Hoemann, Montreal (CA); Catherine Marchand, Montreal (CA)

Correspondence Address: CANTOR COLBURN, LLP, 20 Church Street, 22nd Floor, Hartford, CT, 06103, US

(21) Appl. No.: 12/516,900

(22) PCT Filed: Nov. 30, 2007
(86) PCT No.: PCT/CA2007/002163

§ 371 Date: Feb. 10, 2010

Related U.S. Application Data
(60) Provisional application No. 60/867,959, filed on Nov. 30, 2006.

(51) Int. Cl. A61K 35/14; A61K 38/18; A61P 19/04

(52) U.S. Cl. 424/532; 424/529

(57) ABSTRACT

The present invention relates to a method for repairing or regenerating tissues in a patient such as cartilage, meniscus, ligament, tendon, bone, skin, cornea, periodontal tissues, abscesses, resected tumors, cardiac tissues and ulcers. The method comprises the step of administering simultaneously or sequentially a pro-coagulant factor and an effective amount of a polymer composition comprising a biocompatible polymer and blood or a component thereof. When the polymer composition is in contact with the pro-coagulant factor it is converted into a non-liquid state such that the polymer composition will adhere to the site in need of repair to effect repair of the tissue and/or regeneration thereof.

### BACKGROUND OF THE INVENTION

(a) Field of the Invention

The invention relates to a method for inducing in situ-solidification of blood containing polymers on wounds or surgical defects. The resulting solid implants stimulate the repair and regeneration of articular cartilage, joint tissues and other tissues including meniscus, ligament, tendon, bone, skin, cornea, periodontal tissues, abscesses, resected tumors, ulcers, aorta, and cardiac tissue.

(b) Description of Prior Art

1) The Cartilage Repair Problem:

Cartilage: Structure, Function, Development, Pathology

Articular cartilage covers the ends of bones in diarthroidial joints in order to distribute the forces of locomotion to underlying bone structures while simultaneously providing nearly frictionless articulating interfaces.

Articular cartilage is formed during the development of long bones following the condensation of prechondrocytic mesenchymal cells and induction of a phenotype switch from predominantly collagen type I to collagen type II and aggrecan. Bone is formed from cartilage when chondrocytes hypertrophy and switch to type X collagen expression, accompanied by blood vessel invasion, matrix calcification, the appearance of osteoblasts and bone matrix production. In the adult, a thin layer of articular cartilage remains on the ends of bones and is sustained by chondrocytes through synthesis, assembly and turnover of extracellular matrix. Articular cartilage disease arises when fractures occur due to physical trauma or when a more gradual erosion, as is characteristic of many forms of arthritis, exposes subchondral bone to create symptomatic joint pain. In addition to articular cartilage, cartilaginous tissues remain in the adult at several body sites such as the ears and nose, areas that are often subject to reconstructive surgery.

2) Cartilage Repair: the Natural Response

Articular cartilage has a limited response to injury in the adult mainly due to a lack of vascularisation and the presence of a dense proteoglycan rich extracellular matrix. The former inhibits the appearance of inflammatory and pluripotential repair cells, while the latter emprisons resident chondrocytes in a matrix non-conducive to migration. However, lesions that penetrate the subchondral bone create a conduit to the highly vascular bone allowing for the formation of a fibrin clot that traps cells of bone and marrow origin in the lesion leading to a granulation tissue. The deeper portions of the granulation tissue reconstitute the subchondral bone plate while the upper portion transforms into a fibrocartilagenous repair tissue. This tissue can temporarily possess the histological appearance of hyaline cartilage although not its mechanical properties and is therefore unable to withstand the local mechanical environment leading to the appearance of degeneration before the end of the first year post-injury. Thus the natural response to repair in adult articular cartilage is that partial thickness lesions have no repair response (other than cartilage flow and localized chondrocyte cloning) while full-thickness lesions with bone penetration display a limited and failed response. Age, however, is an important factor since full thickness lesions in immature articular cartilage heal better than in the adult, and superficial lacerations in fetal articular cartilage heal completely in one month without any involvement of vasculature or bone-derived cells.

3) Current Approaches for Assisted Cartilage Repair

Current clinical treatments for symptomatic cartilage defects involve techniques aimed at: 1) removing surface irregularities by shaving and debridement 2) penetration of subchondral bone by drilling, fracturing or abrasion to augment the natural repair response described above (i.e. the family of bone-marrow stimulation techniques) 3) joint realignment or osteotomy to use remaining cartilage for articulation 4) pharmacological modulation 5) tissue transplantation and 6) cell transplantation. Most of these methods have been shown to have some short term benefit in reducing symptoms (months to a few years), while none have been able to consistently demonstrate successful repair of articular lesions after the first few years. The bone marrow-stimulation techniques of shaving, debridement, drilling, fracturing and abrasion athroplasty permit temporary relief from symptoms but produce a sub-functional fibrocartilagenous tissue that can be readily degraded under normal daily load-bearing. In a 5-year follow-up, 10 out of 40 patients treated with microfracture were considered failures in need of total knee arthoplasty.

Pharmacological modulation supplying growth factors to defect sites can augment natural repair but to date insufficiently so. Allograft and autograft osteochondral tissue transplants containing viable chondrocytes can effect a more successful repair but suffer from severe donor limitations, especially in degenerated joints.

There is no universal consensus among orthopaedists on which type of articular cartilage lesion should receive which type of treatment. There is also a lack of rigorous scientific studies that demonstrate the efficacy of these treatments for particular indications. Thus the choice of treatment for cartilage lesions is largely dependent on the training, inclinations and personal experience of the practitioner. Reasons for this lack of consensus are multifold but include the variability in the type of lesion treated and a variable if not uncontrolled success in the formation of a “good quality” blood clot.

Bleeding and the resulting formation of a blood clot initiate the cartilage repair following marrow stimulation. Coagulation is the biological initiator of spontaneous wound repair (Clark, R. A., The molecular and cellular biology of wound repair, Arch Dermatol, 132:1531, 1996. Notes: 2nd Ed, New York: Plenum). Following tissue damage, whole blood will leak into the extravascular space and trigger the extrinsic clotting cascade (Colman R W, Clowes A W, George J N, Hirsh, J, Marder V J, Chapter 1, Overview of Hemostasis, In: Hemostasis and Thrombosis, Basic Principles & Clinical Practice, Lippincott Williams & Wilkins, Fourth Ed. 2001). Coagulation is initiated when a small amount of circulating Factor VIIa in the plasma comes in contact with its extravascular ligand Tissue Factor (TF) which is a transmembrane receptor enzyme expressed on the surface of vascular smooth muscle and connective tissue cells. The TF-VIIa (extrinsic tenase) enzyme complex generates Factor Xa, a pro-thrombinase enzyme with a gla domain. Factor Xa and prothrombin (Factor II) both harbour negatively charged gla domains which tether these factors via calcium to negatively charged phospholipid substrates such as flip-flop membranes of activated platelets. The pro-thrombinase complex includes Factor Xa, its non-enzymatic cofactor, Factor Va, calcium, and phospholipids. Extrinsically generated Factor Xa by-passes the need for Factors VIII and IX, factors involved in the formation of the intrinsic tenase complex. Pro-thrombinase is a powerful thrombin-generating assembly giving rise to a local burst activation of thrombin at the surface of activated platelets. Thrombin simultaneously activates platelets, converts fibrinogen (Factor I) to fibrin and activates Factor XIIIa, a plasma transglutaminase involved in covalent cross-linkage of polymerized fibrin monomer. Thrombin activation, fibrin polymerization and fibrin cross-linkage by Factor XIIIa is termed the common pathway. Polymerization of a cross-linked fibrin network in whole blood leads to the development of a clot tensile strength which can be measured by a method such as thromboelastography. Polymerized fibrin is anchored to the platelet surface via fibrin-integrin receptor interactions which subsequently permits clot retraction via an energy-dependent actin-myosin motor apparatus linked to the platelet integrin receptor.

Coagulation leads to the activation of clotting factors and the release of soluble wound-repair factors from platelets and leukocytes, all of which stimulate important wound-repair responses such as cell survival, chemotaxis, mitogenesis (Fan L, Yotov W V, Zhu T, Esmailzadeh L, Joyal J S, Sennulaub F, Heveker N, Chemtob S, Rivard G E, Tissue factor enhances protease-activated receptor-2-mediated factor VIIa cell proliferative properties, J. Thrombosis and Hemostasis, 3 (5):1056-1063 May 2005) and angiogenesis (Clark R. A., The molecular and cellular biology of wound repair, Arch Dermatol, 132:1531, 1996. Notes: 2nd ed, New York: Plenum). Lysis and clearance of the blood clot by endogenous proteases and phagocytes promote additional wound-repair responses through the timed and gradual release of phagocyte-derived factors and clot debris including plasmin fragments, hemolyzed erythrocytes, and fibrin fragments (Clark R. A., The molecular and cellular biology of wound repair, Arch Dermatol, 132:1531, 1996. Notes: 2nd ed, New York: Plenum).

US application Serial No. 2005/0244393 discloses a sealant or a tissue generating product comprising a (coagulated) plasma matrix, one or more growth factors, at least one phospholipid and a protein scaffold for the generation of said tissue (or the coagulation factor VII).

US application Serial No. 2006/008524 discloses a composition comprising nanoscale particles comprising tissue factor or recombinant tissue factor, a membrane scaffold protein and phospholipid, for controlling bleeding in a human or animal patient.

Some of the problems associated with forming a good quality blood clot following cartilage repair procedures are 1) the uncontrolled nature of the bleeding coming from the bone, which never fills up the cartilage lesion entirely 2) platelet mediated clot contraction occurring within minutes of clot formation reduces clot size and could detach it from surrounding cartilage 3) dilution of the bone blood with synovial fluid or circulating arthroscopy fluid and 4) the fibrinolytic or clot dissolving activity of synovial fluid. Some of these issues were the motivation behind some studies where a blood clot was formed ex vivo and then cut to size and packed into a meniscal defect or an osteochondral defect (Paletta, G. A., S. P. Amoczky, and R. F. Warren, “The repair of osteochondral defects using an exogenous fibrin clot. An experimental study in dogs”, Am J Sports Med 20 (6):725-31, 1992). Something similar to the classical wound healing cascade then ensued to aid healing of the defect. This approach did clearly provide more filling of the defect with repair tissue, however the quality of the repair tissue was generally not acceptable, being predominantly fibrous and mechanically insufficient. Some probable reasons for a less than satisfactory repair tissue with this approach are 1) continued platelet mediated clot contraction 2) the lack of viability of some blood components due to extensive ex vivo manipulation and 3) the solidification of the clot ex vivo which precludes good adhesion to all tissue surfaces surrounding the cartilage defect and limits defect filling. In summary, current clinical procedures practised by orthopaedists for treating focal lesions of articular cartilage mostly depend on the formation of a blood clot within the lesion. However the ability to form a good quality blood clot that fills and adheres to the lesion and contains all of the appropriate elements for wound healing (platelets, monocytes, fibrin network etc) in a viable state produces inconsistent and often unsatisfactory outcomes.

4) Repair of Other Tissues Including Meniscus, Ligament, Tendon, Bone, Skin, Cornea, Periodontal Tissues, Abscesses, Resected Tumours, Ulcers, and Cardiac Tissue

Natural and assisted repair of musculoskeletal and other tissues are very broad fields with numerous complex biological processes and a wide variety of approaches to accelerate the repair process (as in bone repair), aid it in tissues that have little intrinsic repair capacity (as in cartilage repair), and to reduce scarring (as in burn treatments) (Clark, Richard A. F., The molecular and cellular biology of wound repair, Arch. Dermatol, 132:1531, 1996, 2 ed. New York: Plenum). Although differences certainly occur in the biological elements and processes involved, the global events in (non-fetal) wound repair are identical. These include the formation of a blood clot at the site of tissue disruption, release of chemotactic and mitogenic factors from platelets, influx of inflammatory cells and pluripotential repair cells, vascularisation, and finally the resolution of the repair process by differentiation of repair cells and the synthesis of extracellular matrix components. In a successful repair outcome the specific local tissue environment and the specific local population of pluripotential repair cells will lead to the formation of the correct type of tissue, bone to replace bone, skin to replace skin etc. Given the similarity of the general elements in the tissue repair process, it is not surprising that approaches to aid repair in one tissue could also have some success in aiding repair in other tissues.

This possibility becomes much more likely if the method and composition to aid repair is based upon augmenting some aspect of the natural wound healing cascade without significantly deviating from this more or less optimised sequence of events.

However similar problems associated with forming a good quality blood clot are observed.

5) Use of Chitosan in Pharmaceuticals, Wound Healing, Tissue Repair and as a Hemostatic Agent

Chitosan, which primarily results from the alkaline deacetylation of chitin, a natural component of shrimp and crab shells, is a family of linear polysaccharides that contains 1-4 linked glucosamine (predominantly) and N-acetyl-glucosamine monomers. Chitosan and its amino-substituted derivatives are pH-dependent, bioerodible and biocompatible cationic polymers that have been used in the biomedical industry for wound healing and bone induction (Shigemasa, Y., and S. Minami, Applications of chitin and chitosan for biomaterials. Biotechnol Genet Eng Rev 13:383-420, 1996). Chitosan is termed a mucoadhesive polymer since it adheres to the mucus layer of the gastrointestinal epithelia via ionic and hydrophobic interactions, thereby facilitating per oral drug delivery. Biodegradability of chitosan occurs via its susceptibility to enzymatic cleavage by a broad array of endogenous enzymes including chitinases, lysozymes, cellulases and lipases (Shigemasa, Y., and S. Minami, Applications of chitin and chitosan for biomaterials. Biotechnol Genet Eng Rev 13:383-420, 1996). Recently, chondrocytes have been shown to be capable of expressing chitotriosidase, the human analogue of chitosanase; its physiological role may be in the degradation of hyaluronan, a linear polysaccharide possessing some similarity with chitosan since it is composed of disaccharides of N-acetyl-glucosamine and glucuronic acid.

The properties of chitosan that are most commonly cited as beneficial for the wound repair process are its biodegradability, adhesiveness, prevention of dehydration and as a barrier to bacterial invasion. Other properties that have also been claimed are its cell activating and chemotractant nature (Shigemasa, Y., and S. Minami. 1996. Applications of chitin and chitosan for biomaterials. Biotechnol Genet Eng Rev 13:383-420) its hemostatic activity (Malette, W. G., and H. J. Quigley, 1985, “Method of achieving hemostasis, inhibiting fibroplasia, and promoting tissue regeneration in tissue wound.” U.S. Pat. No. 4,532,134) and an apparent ability to limit fibroplasia and scarring by promoting a looser type of granulation tissue.

Chitosan has been proposed in various formulations, alone and with other components, to stimulate repair of dermal, corneal and hard tissues in a number of reports and inventions.

Therefore, to enhance the osteochondral repair response following marrow stimulation, a wound-stimulatory implant has been developed, consisting of an autologous, in situ solidifying scaffold-stabilized blood clot (Hoemann, C. D.; Hurtig, M.; Rossomacha, E.; Sun, J.; Chevrier, A.; Shive, M. S.; and Buschmann, M. D.: Chitosan-glycerol phosphate/blood implants improve hyaline cartilage repair in ovine microfracture defects. J Bone Joint Surg Am, 87:2671-86, 2005). The scaffold-stabilized clot is generated by mixing a cytocompatible polymer solution such as glycerol phosphate-buffered chitosan with unclotted whole blood (WO 02/00272) (Hoemann, C. D., Hurtig, M., Rossomacha, E., Sun, J., Chevrier, A., Shive, M. S., and Buschmann, M. D.: Chitosan-glycerol phosphate/blood implants improve hyaline cartilage repair in ovine microfracture defects. J Bone Joint Surg Am, 87:2671-86, 2005; Hoemann, C. D., Sun, J., McKee, M. D., Chevrier, A., Rossomacha, E., Rivard, G. E., Hurtig, M., and Buschmann, M. D.: Chitosan-glycerol phosphate/blood implants elicit hyaline cartilage repair integrated with porous subchondral bone in microdrilled rabbit defects. Osteoarthritis Cartilage, 2007, 15 (1) 78-89). Application of the implant to cartilage defects with channels to the subchondral bone marrow (marrow stimulation) stimulates revascularization and active bone remodeling of the subchondral bone and calcified cartilage layer (Hoemann, C. D., Sun, J., McKee, M. D., Chevrier, A., Rossomacha, E., Rivard, G. E., Hurtig, M., and Buschmann, M. D.: Chitosan-glycerol phosphate/blood implants elicit hyaline cartilage repair integrated with porous subchondral bone in microdrilled rabbit defects, Osteoarthritis Cartilage, 2007, 15 (1) 78-89; Chevrier, A.; Hoemann, C. D.; Sun, J.; and Buschmann, M. D.: Chitosan-glycerol phosphate/blood implants increase cell recruitment, transient vascularization and subchondral bone remodeling in drilled cartilage defects. Osteoarthritis Cartilage, 2007, 15 (3) 316-27,) which in turn leads to the formation of a more hyaline cartilage repair tissue compared to marrow stimulation alone (Hoemann, C. D.; Hurtig, M.; Rossomacha, E.; Sun, J.; Chevrier, A.; Shive, M. S.; and Buschmann, M. D.: Chitosan-glycerol phosphate/blood implants improve hyaline cartilage repair in ovine microfracture defects. J Bone Joint Surg Am, 87: 2671-86, 2005. Hoemann, C. D.; Sun, J.; McKee, M. D.; Chevrier, A.; Rossomacha, E.; Rivard, G. E.; Hurtig, M.; and Buschmann, M. D.: Chitosan-glycerol phosphate/blood implants elicit hyaline cartilage repair integrated with porous subchondral bone in microdrilled rabbit defects. Osteoarthritis Cartilage, 2007, 15 (1) 78-89). The hyaline cartilage repair tissue elicited by chitosan-GP/blood implants in the load-bearing region of the medial femoral condyle after 6 months in a large animal model contained the same average level of glycosaminoglycan as native cartilage, 49 mg/g, while microfracture-only controls contained much lower average GAG levels, 27 mg/g wet weight (Hoemann, C. D.; Hurtig, M.; Rossomacha, E.; Sin, J.; Chevrier, A.; Shive, M. S.; and Buschmann, M. D.: Chitosan-glycerol phosphate/blood implants improve hyaline cartilage repair in ovine microfracture defects. J Bone Joint Surg Am, 87: 2671-86, 2005). Relative to microfracture-only controls, microfracture defects treated with chitosan-GP/blood implant had a higher average percent fill with repair tissue compared to the original cartilage volume (52% treated vs 31% control) and repair cartilage with significantly greater hyaline quality (86% vs 71% control). In the best case repair, a zonal organization of the repair cartilage tissue that resembled normal articular cartilage was seen (Buschmann M. D.; Hoemann, C. D.; Hurtig, M.; Shive, M. S.; Strategies in Cartilage Repair. In Cartilage repair with chitosan/glycerol-phosphate stabilised blood clots, Edited by Riley J Williams Humana Press, 2006), suggesting that appositional processes active during normal organogenesis were stimulated by the chitosan-GP/blood implant. The chitosan-GP/blood implant could be used to stimulate regeneration of a wide variety of damaged tissues.

Chitosan-GP/blood implants which are composed of 3 parts whole blood and one part liquid polymer solution solidify within ten minutes of depositing in a surgical defect.

The time for the surgeon to administer this therapy is about fifteen minutes.

The time of administration and solidification of ten to fifteen minutes is relatively long in an operating unit with respect to the duration where the patient's tissue is uncovered and to the duration of occupation of the operation unit. It would be advantageous to reduce this time to more practical time in order to limit the risks of infections and to treat more patients for the same duration of occupation of the operation unit.

6) Summary of Prior Art

In summary of prior art for assisted cartilage repair, it may be said that many techniques to improve the very limited natural repair response of articular cartilage have been proposed and experimentally tested. Some of these techniques have achieved a certain level of acceptance in clinical practice but this has mainly been so due to the absence of any practical and clearly effective method of improving the repair response compared to that found when the family of bone marrow stimulation techniques is applied. Chitosan-GP/blood implants combined with proper surgical technique increase the generation of hyaline repair tissue over surgical technique alone. The implant is solidified in situ, in order to establish an optimally adhesive contact between the implant and the surface of the treated lesion. In situ delivery entails applying the liquid mixture onto the lesion followed by implant solidification in the lesion. In cartilage repair models involving live animals and in clinical applications with human patients, the chitosan-GP/blood implant reproducibly solidifies in situ within 10 minutes, requiring the surgeon to wait almost 15 minutes before terminating the operation. Chitosan has been identified as a polymer that stimulates revascularization and repair of marrow-stimulated defects. Other substances that mimic processes identified as playing a role in chitosan-GP/blood-mediated bone and cartilage repair, such as subchondral angiogenesis, macrophage and stem cell chemotaxis, and bone remodeling, could also be used to stimulate wound repair, provided these factors or polymers are immobilized at the site in need of repair or regeneration, in a hybrid blood clot in a practical time.

### SUMMARY OF THE INVENTION

At the time of making this invention, the inventors postulated that a method that permits a more rapid solidification of the injected liquid implant in the lesion which would shorten the length of the surgical procedure would be highly desirable. Solidification of the composition should be controlled so as not to induce solidification of the liquid implant in the mixing vial or delivery syringe. The formulation which solidifies rapidly in situ should generate an equal or improved wound-repair response, compared to the chitosan-GP/blood implant (Hoemann, C. D.; Sun, J.; McKee, M. D.; Chevrier, A.; Rossomacha, E.; Rivard, G. E.; Hurtig, M.; and B Buschmann, M. D.: Chitosan-glycerol phosphate/blood implants elicit hyaline cartilage repair integrated with porous subchondral bone in microdrilled rabbit defects. Osteoarthritis Cartilage, 2007, 15 (1) 78-89); Chevrier, A.; Hoemann, C. D.; Sun, J.; and Buschmann, M. D.: Chitosan-glycerol phosphate/blood implants increase cell recruitment, transient vascularization and subchondral bone remodeling in drilled cartilage defects. Osteoarthritis Cartilage, 2007, 15 (3) 316-27).

The present invention provides a method for repair and/or regeneration of tissues.

In accordance with one aspect of the present invention, there is thus provided a method for repair, regeneration, reconstruction or bulking of tissues of cartilaginous tissues or other tissues such as meniscus, ligament, tendon, bone, skin, cornea, periodontal tissues, abscesses, resected tumors, cardiac tissues, and ulcers in a patient.

In accordance with one aspect of the present invention, there is thus provided a method for repair and/or regeneration of a tissue of a patient comprising administering into said tissue in need of repair and/or regeneration a pro-coagulant factor and a polymer composition comprising a biocompatible polymer and blood or a blood component thereof. When the polymer composition is in contact with the pro-coagulant factor, it is converted into a non-liquid state such that the polymer composition when placed at the site in need of repair and/or regeneration will adhere to the site in need of repair to effect repair of the tissue and/or regeneration thereof.

In accordance with one aspect of the present invention, there is thus provided a method for repair and/or regeneration in a tissue of a patient comprising administering into said tissue in need of repair and/or regeneration a pro-coagulant factor and a polymer composition comprising chitosan, glycerol phosphate and blood or blood component thereof. When the polymer composition is converted into a non-liquid state in time, the composition once converted into a non-liquid state adheres to the site in need of repair and/or regeneration when placed thereon to effect repair and/or regeneration of the tissue.

In accordance with another aspect of the present invention, there is thus provided a method for repair and/or regeneration in a tissue of a patient comprising administering simultaneously or sequentially into said tissue in need of repair a pro-coagulant factor and blood mixed with an effective amount of a factor capable of stimulating biological reactions that improve the spontaneous repair response, including but not limited to cell chemotaxis, angiogenesis, macrophage chemoattraction, stem cell chemotaxis, and cell survival. The composition would also be improved by the presence of components that aid in adhesion of the blood clot to the site in need of repair or regeneration.

In accordance with another aspect of the invention, there is thus provided a kit for repair and/or regeneration of a tissue of a patient, said kit comprising i) a pro-coagulant factor and ii) a polymer composition comprising a biocompatible polymer.

In accordance with another aspect of the invention, there is thus provided a kit for repair and/or regeneration in a tissue of a patient comprising i) a pro-coagulant factor and ii) a polymer composition comprising chitosan and glycerol phosphate.

In accordance with another aspect of the invention, there is thus provided a use of a pro-coagulant factor and of a polymer composition comprising i) a biocompatible polymer; and ii) blood or a component thereof, for repairing and/or regenerating a tissue of a patient, wherein the polymer composition in contact with the pro-coagulant factor is converted into a non-liquid state such that the polymer composition when placed at the site in need of repair will adhere to the site in need of repair to effect repair of the tissue and/or regeneration thereof.

In accordance with another aspect of the invention, there is thus provided a use of a pro-coagulant factor and of a polymer composition comprising i) a biocompatible polymer; and ii) blood or a component thereof, in the manufacture of a medicament for repairing and/or regenerating a tissue of a patient, wherein the polymer composition in contact with the pro-coagulant factor is converted into a non-liquid state such that the polymer composition when placed at the site in need of repair will adhere to the site in need of repair to effect repair of the tissue and/or regeneration thereof. Use of a pro-coagulant factor and of a polymer composition comprising chitosan, glycerol phosphate and blood or a component of said blood for repairing and/or regenerating a tissue of a patient, wherein the polymer composition is converted into a non-liquid state in time or upon heating, said composition once converted into a non-liquid state adheres to the site in need of repair when placed thereon to effect reconstruction or bulking of the tissue and/or regeneration thereof.

In accordance with another aspect of the invention, there is thus provided a use of a pro-coagulant factor and of a polymer composition comprising chitosan, glycerol phosphate and blood or a component of said blood for repairing and/or regenerating a tissue of a patient, wherein the polymer composition is converted into a non-liquid state in time or upon heating, said composition once converted into a non-liquid state adheres to the site in need of repair when placed thereon to effect reconstruction or bulking of the tissue and/or regeneration thereof.

In accordance with another aspect of the invention, there is thus provided a use of a pro-coagulant factor and of a polymer composition comprising chitosan, glycerol phosphate and blood or a component of said blood in the manufacture of a medicament for repairing and/or regenerating a tissue of a patient, wherein the polymer composition is converted into a non-liquid state in time or upon heating, said composition once converted into a non-liquid state adheres to the site in need of repair when placed thereon to effect reconstruction or bulking of the tissue and/or regeneration thereof.

### BRIEF DESCRIPTION OF THE DRAWINGS

FIG. 1 represents the evaluation of the solidification time and tensile strength for whole unmodified blood with and without tissue Plasminogen Activator, tPA (FIG. 1A), Chitosan-GP/blood with and without tissue Plasminogen Activator, tPA (FIG. 1B), whole unmodified blood with and without thrombin (IIa, FIG. 1C), chitosan-GP/blood with and without IIa (FIGS. 1D & 1E), chitosan-GP/blood with rVIIa (FIG. 1E), chitosan-GP/blood with rVIIa and Tissue Factor (TF) (FIG. 1E), chitosan-GP/blood with and without TF (FIGS. 1E & 1F), Chitosan-GP/blood (CG) with and without TF, TF-rVIIa and/or rVIIa, and GP/blood alone (FIG. 1G).

FIG. 2 is a graphical representation of the quantification of levels of thrombin generation via serum thrombin-antithrombin (TAT) complex levels in chitosan-GP/blood, GP/blood, and whole blood.

FIG. 3 represents a multivariate analysis demonstrating the correlation between thrombin generation and solidification of both whole blood and chitosan-GP/blood.

FIG. 4 represents the Western blots of serum samples showing platelet activation.

FIG. 5 represents the Western blots of serum samples showing Factor XIII activation.

FIG. 6 shows a model explaining how clotting factors accelerate solidification of polymer-whole blood mixtures.

FIG. 7 represents photographs of the ex vivo cartilage defects receiving the samples: Chitosan-GP (7A), chitosan-GP/blood homogenously mixed with rVIIa (7B), chitosan-GP mixed homogenously with IIa then mixed with whole blood (7C).

FIG. 8 represents the clot formation of various combinations of the polymer compositions mixed homogenously with or without clotting factors in plastic or glass vials.

FIG. 9 represents the application of distinct clotting factors on a glass or plastic Petri, followed by 2 to 3 drops of rabbit whole blood.

FIG. 10 represents the co-application of whole blood or blood-polymer mixture and a clotting factor.

FIG. 11 represents the in vivo studies in rabbits.

FIG. 12 is a graphical representation of the in situ solidification time of chitosan-GP/blood implants with or without additional clotting factor in live animals.

FIG. 13 represents photographs of the repaired rabbit tissues.

FIG. 14 represents the histology of repaired rabbit defects after 3 weeks of repair.

FIG. 15 represents histology of repaired rabbit defects at 8 weeks post-repair.

FIG. 16 represents a schema of the invention.

FIG. 17 represents another schema of the invention.

FIG. 18 represents another schema of the invention.

### DETAILED DESCRIPTION OF THE INVENTION

The present invention provides a method for repair and/or regeneration of tissues.

In accordance with one aspect of the present invention, there is thus provided a method for repair, regeneration, reconstruction or bulking of tissues of cartilaginous tissues or other tissues such as meniscus, ligament, tendon, bone, skin, cornea, periodontal tissues, abscesses, resected tumors, ulcers or cardiac tissues in a patient.

In accordance with one aspect of the present invention, there is thus provided a method for repair and/or regeneration of a tissue of a patient comprising administering into said tissue in need of repair a pro-coagulant factor and a polymer composition comprising a biocompatible polymer and blood or a blood component thereof. When the polymer composition is in contact with the pro-coagulant factor, it is converted into a non-liquid state such that the polymer composition when placed at the site in need of repair will adhere to the site in need of repair to effect repair of the tissue and/or regeneration thereof.

In one embodiment, the polymer composition is administered in an effective amount.

In one embodiment, the pro-coagulant factor and the polymer composition are administered simultaneously. In one embodiment, the pro-coagulant factor and the polymer composition are administered sequentially. In one embodiment, the pro-coagulant factor is administered before the polymer composition. In one embodiment, the pro-coagulant factor is administered after the polymer composition. In one embodiment, the pro-coagulant factor is administered to the tissue immediately prior to or with a delay of up to 2 minutes, before administering the polymer composition. In one embodiment, the pro-coagulant factor is administered to the tissue immediately after to or with a delay of up to 2 minutes, after administering the polymer composition. In another embodiment, the polymer composition is converted into a non-liquid state through enzymatic activity of the pro-coagulant factors, with or without gelation of the polymer.

In another embodiment, the polymer composition is solidifying.

In another embodiment, the polymer composition when placed at the site in need of repair will solidify.

In another embodiment, the polymer composition is thermogelling.

In accordance with one aspect of the present invention, the biocompatible polymer is selected from the group consisting of a polysaccharide, a protein, a lipid, a nucleic acid, and a polyamino acid.

In another embodiment, the polysaccharide is a modified or natural polysaccharide. In another embodiment, the polysaccharide is selected from the group consisting of chitosan, chitin, hyaluronan, glycosaminoglycan, chondroitin sulfate, keratan sulfate, dermatan sulfate, heparin, cellulose, and heparin sulfate.

In another embodiment, the polysaccharide is chitosan.

In another embodiment, the polymer composition is dissolved in an organic or inorganic phosphate buffer, such as a phosphate or glycerol phosphate containing buffer.

In another embodiment, the biocompatible polymer is dissolved in an organic or inorganic phosphate buffer, such as a phosphate or glycerol phosphate containing buffer.

In another embodiment, the chitosan is dissolved in an organic or inorganic phosphate buffer, such as a phosphate or glycerol phosphate containing buffer.

In another embodiment, the chitosan in the polymer composition is in a soluble state, with the polymer composition having a pH between 6.2 and 7.8.

In another embodiment, the chitosan in the polymer composition is in a soluble state, with the polymer composition having a pH between 6.5 and 7.4.

In another embodiment, the chitosan in the polymer composition is in a soluble state without addition of buffer, with a salt content adjusted to near isotonicity (200 to 600 mOsm).

In another embodiment, the protein is a natural, recombinant or synthetic protein. In one embodiment, the natural protein is soluble collagen or gelatin.

In another embodiment, the protein is a polyamino acid, such as polylysine.

In another embodiment, the polymer is a nucleic acid, either double-strand DNA, single strand DNA, RNA, or a short interfering RNA (siRNA), with or without a complexing agent.

In another embodiment, the blood or the blood component thereof is autologous or non-autologous to the patient. In another embodiment, the blood or the blood component thereof is autologous to the patient. In another embodiment, the blood or the blood component thereof is non-autologous to the patient.

In another embodiment, the blood may be for example without limitation whole blood, processed blood, venous blood, arterial blood, blood from bone-marrow, umbilical cord blood and placenta blood. The blood may also be enriched in platelets.

In another embodiment, the blood component is selected from the group consisting of erythrocytes, leukocytes, monocytes, platelets, fibrinogen, and thrombin. The blood component may comprise platelet rich plasma free of erythrocytes.

In another embodiment, the polymer composition further comprises a growth factor.

In another embodiment, the polymer composition further comprises a macrophage chemotactic factor. In another embodiment, the macrophage chemotactic factor is selected from the group consisting of Macrophage Chemoattractant Protein-1 (MCP-1), Macrophage Inflammatory Protein-1 (MIP-1alpha, MIP-1beta), Interleukin 8 (IL-8), Eotaxin, EGF, and G-CSF.

In another embodiment, the polymer composition further comprises a stem cell chemotactic factor.

In another embodiment, the polymer composition further comprises a cell survival factor.

In another embodiment, the polymer composition, the biocompatible polymer or the chitosan are dissolved or suspended in a buffer containing organic or inorganic salts. In another embodiment, the organic salts are selected from the group consisting of glycerol-phosphate, fructose phosphate, glucose phosphate, L-serine phosphate, adenosine phosphate, glucosamine, galactosamine, HEPES, PIPES and MES. In another embodiment, the inorganic salts are selected from the group consisting of sodium chloride or phosphates, sulfates or carboxylates of potassium, calcium and magnesium.

Glycerol-phosphate includes beta-glycerol-phosphate and alpha-glycerol-phosphate, but beta-glycerol-phosphate is preferred.

In another embodiment, the polymer composition has a pH between 6.2 and 7.8.

In another embodiment, the polymer composition has a pH between 6.5 and 7.4.

In another embodiment, the polymer composition has an osmolarity adjusted to a physiological value between 250 mOsm/L and 600 mOsm/L.

In another embodiment, the polymer composition is used in a ratio varying from 1:100 to 100:1 volume to volume with respect to the blood or blood component thereof. In another embodiment, the polymer composition is used in a ratio 1:10 volume to volume with respect to the blood or blood component thereof. In another embodiment, the polymer composition is used in a ratio 1:20 volume to volume with respect to the blood or blood component thereof. In another embodiment, the polymer composition is used in a ratio 1:30 volume to volume with respect to the blood or blood component thereof. In another embodiment, the polymer composition is used in a ratio 1:40 volume to volume with respect to the blood or blood component thereof. In another embodiment, the polymer composition is used in a ratio 1:50 volume to volume with respect to the blood or blood component thereof. In another embodiment, the polymer composition is used in a ratio 1:60 volume to volume with respect to the blood or blood component thereof. In another embodiment, the polymer composition is used in a ratio 1:70 volume to volume with respect to the blood or blood component thereof. In another embodiment, the polymer composition is used in a ratio 1:80 volume to volume with respect to the blood or blood component thereof. In another embodiment, the polymer composition is used in a ratio 1:90 volume to volume with respect to the blood or blood component thereof.

In another embodiment, the polymer and blood or component thereof are mechanically mixed using sound waves, stirring, vortexing, shaking, or multiple passes in syringes.

In another embodiment, the pro-coagulant factor is selected from the group consisting of collagen, ellagic acid, epinephrine and adenosine diphosphate.

In another embodiment, the pro-coagulant factor is a platelet-activating factor. In another embodiment, the platelet-activating factor is selected from the group consisting of arachidonic acid, ADP, collagen, Thromboxane A2 and 5-HT.

In another embodiment, the pro-coagulant factor is a clotting factor, such as thrombin, factor VIIa, tissue factor, factor XIII, factor XIIIa, Factor IX, Factor X, Factor Xa, Factor XIa, Factor V, Factor Va, Factor VII, rVIIa, fibrinogen, fibrin, phospholipids, phosphatidyl serine, phosphatidyl choline, phosphatidyl inositol, phosphoryl choline, calcium, tissue factor-phospholipids, tissue factor ectodomain, tissue factor ectodomain—phospholipids, tissue factor ectodomain—phospholipids-rVIIa and tissue factor-phospholipids-rVIIa.

In another embodiment, the pro-coagulant factor is dissolved in a buffer containing calcium.

In another embodiment, the thrombin is activated.

In another embodiment, the concentration of thrombin is between 0.001 U/mL and 1000 U/mL. In another embodiment, the concentration of thrombin is between 0.01 U/mL and 100 U/mL. In another embodiment, the concentration of thrombin is between 0.1 U/mL and 10 U/mL.

In another embodiment, the concentration of tissue factor is between 0.01 pM and 100 nM. In another embodiment, the concentration of tissue factor is between 0.1 pM and 10 nM. In another embodiment, the concentration of tissue factor is between 1 pM and 1 nM.

In another embodiment, the concentration of tissue factor is between 0.1 pg/mL and 10 μg/mL. In another embodiment, the concentration of tissue factor is between 1 pg/mL and 1 μg/mL.

In another embodiment, the concentration of rVIIa is between 50 pg/mL and 500 μg/mL. In another embodiment, the concentration of rVIIa is between 500 pg/mL and 50 μg/mL. In another embodiment, the concentration of XIIIa is between 0.01 and 100 U/mL. In another embodiment, the concentration of XIIIa is between 0.1 and 10 U/mL.

In another embodiment, Factor XIII or XIIIa promotes implant cross-linking.

In another embodiment, the phospholipids are phosphoryl choline, phosphatidyl choline, phosphatidyl inositol, or phosphatidyl serine.

In another embodiment, the pro-coagulant factor is in a volume ratio 1:1-100 volumes pro-coagulant factor to polymer composition. In another embodiment, the pro-coagulant factor is in a volume ratio 1:20 volumes pro-coagulant factor to polymer composition. In another embodiment, the pro-coagulant factor is in a volume ratio 1:30 volumes pro-coagulant factor to polymer composition. In another embodiment, the pro-coagulant factor is in a volume ratio 1:40 volumes pro-coagulant factor to polymer composition. In another embodiment, the pro-coagulant factor is in a volume ratio 1:50 volumes pro-coagulant factor to polymer composition. In another embodiment, the pro-coagulant factor is in a volume ratio 1:60 volumes pro-coagulant factor to polymer composition. In another embodiment, the pro-coagulant factor is in a volume ratio 1:70 volumes pro-coagulant factor to polymer composition. In another embodiment, the pro-coagulant factor is in a volume ratio 1:80 volumes pro-coagulant factor to polymer composition. In another embodiment, the pro-coagulant factor is in a volume ratio 1:90 volumes pro-coagulant factor to polymer composition.

In another embodiment, administering of the clotting factor to the tissue is done either simultaneously with administration of the polymer composition, or immediately prior to or with a delay of up to 2 minutes, before or after administering the polymer composition.

In another embodiment, administering of the clotting factor to the tissue is done simultaneously with administration of the polymer composition.

In another embodiment, administering of the clotting factor to the tissue is done immediately prior to or with a delay of up to 2 minutes, before administering the polymer composition.

In another embodiment, administering of the clotting factor to the tissue is done immediately after to or with a delay of up to 2 minutes, after administering the polymer composition.

In another embodiment, the method further comprises the administration of cells or additional bioactive factors in the polymer composition or on the tissue of the patient.

The tissue that can be repaired or regenerated is for example without limitation selected from the group consisting of cartilage, meniscus, ligament, tendon, bone, skin, cornea, periodontal tissues, maxillofacial tissues, temporomandibular tissues, abscesses, resected tumors, cardiac tissues and ulcers.

In another embodiment, the tissue is selected from the group consisting of articular cartilage, nose cartilage, ear cartilage, meniscus and avascular cartilage.

In some cases, the site of introduction in the body may be surgically prepared to remove abnormal tissues. Such procedure can be done by piercing, abrading or drilling into adjacent tissue regions or vascularized regions to create channels for the polymer composition to migrate into the site requiring repair. These conduits also serve to remove physical barriers and to facilitate migration of repair cells into the wound area.

In accordance with the present invention, there is also provided a method for repair and/or regeneration in a tissue of a patient comprising administering into said tissue in need of repair a pro-coagulant factor and a polymer composition comprising chitosan, glycerol phosphate and blood or component thereof. When the polymer composition is converted into a non-liquid state in time or upon heating, the composition once converted into a non-liquid state adheres to the site in need of repair when placed thereon to effect repair and/or regeneration of the tissue of the patient.

In another embodiment, this method is intended for reconstruction or bulking of the tissue of a patient.

In another embodiment, the polymer composition is a solidifying composition.

In another embodiment, the polymer composition further comprises a growth factor.

In another embodiment, the polymer composition is dissolved or suspended in a buffer containing organic or inorganic salts.

In another embodiment, the organic salts are selected from the group consisting of glycerol-phosphate, fructose phosphate, glucose phosphate, L-serine phosphate, adenosine phosphate, glucosamine, galactosamine, HEPES, PIPES and MES.

In another embodiment, the inorganic salts are selected from the group consisting of sodium chloride or phosphates, sulfates or carboxylates of potassium, calcium and magnesium.

In another embodiment, the polymer composition has a pH between 6.2 and 7.8. In another embodiment, the polymer composition has a pH between 6.5 and 7.4.

In another embodiment, the polymer composition has an osmolarity adjusted to a physiological value between 250 mOsm/L and 600 mOsm/L. In another embodiment, the polymer composition has an osmolarity adjusted to a physiological value between 300 mOsm/L and 500 mOsm/L.

In another embodiment, the polymer composition is used in a ratio varying from 1:100 to 100:1 volume to volume with respect to the blood, or blood component thereof. In another embodiment, the polymer composition is used in a ratio 1:10 volume to volume with respect to the blood, or blood component thereof. In another embodiment, the polymer composition is used in a ratio 1:20 volume to volume with respect to the blood, or blood component thereof. In another embodiment, the polymer composition is used in a ratio 1:30 volume to volume with respect to the blood, or blood component thereof. In another embodiment, the polymer composition is used in a ratio 1:40 volume to volume with respect to the blood, or blood component thereof. In another embodiment, the polymer composition is used in a ratio 1:50 volume to volume with respect to the blood, or blood component thereof. In another embodiment, the polymer composition is used in a ratio 1:60 volume to volume with respect to the blood, or blood component thereof. In another embodiment, the polymer composition is used in a ratio 1:70 volume to volume with respect to the blood, or blood component thereof. In another embodiment, the polymer composition is used in a ratio 1:80 volume to volume with respect to the blood, or blood component thereof. In another embodiment, the polymer composition is used in a ratio 1:90 volume to volume with respect to the blood, or blood component thereof.

In another embodiment, the polymer composition and blood or component thereof are mechanically mixed using sound waves, stirring, vortexing, shaking, or multiple passes in syringes.

In another embodiment, the chitosan in the polymer composition is in a soluble state, said polymer composition having a pH between 6.2 and 7.8.

In another embodiment, the chitosan in the polymer composition is in a soluble state, said polymer composition having a pH between 6.5 and 7.4.

In another embodiment, the pro-coagulant factor is in a volume ratio 1:1-100 volumes pro-coagulant factor to polymer composition.

In another embodiment, administering of the clotting factor to the tissue is done either simultaneous with administering the solidifying composition, or immediately prior to or after with a delay of up to 2 minutes, before or after administering the polymer composition.

In another embodiment of the invention, the biological elements are based on blood, blood components and additionally isolated cells, both of autologous or non-autologous origin.

The cells may be selected for example from the group consisting of primary cells, passaged cells, selected cells, platelets, stromal cells, stem cells, and genetically modified cells. In one, embodiment, the cells are suspended directly in the blood or blood component, or in a carrier solution, such as a solution containing hyaluronic acid, hydroxyethylcellulose, collagen, alginate, or a water-soluble polymer.

In accordance with the present invention, there is also provided a solidifying chitosan solution for use in culturing cells in vitro, said chitosan solution comprising 0.5-3% w/v of chitosan and being formulated to be solidifying when mixed with blood or a component thereof, said solution being is mixed with whole blood or a component thereof with or without cells prior to being cultured in vitro.

In one embodiment, the polymer composition contains between 0.01 and 10% w/v of 20% to 100% deacetylated chitosan with average molecular weight ranging from 1 kDa to 10 Mda and a blood component.

In another embodiment of the invention, there is provided a kit for repair and/or regeneration of a tissue of a patient.

In another embodiment, there is provided a kit comprising a pro-coagulant factor and a polymer composition comprising a biocompatible polymer.

In another embodiment, the pro-coagulant factor and the polymer composition are in separate containers.

In another embodiment, the polymer composition in the kit further comprises blood or a blood component thereof.

In another embodiment, the polymer composition comprises a pharmaceutical carrier.

Alternatively, this kit comprises i) a polymer composition comprising chitosan, glycerol phosphate, and additionally ii) a pro-coagulant factor.

Alternatively, this kit comprises i) a polymer composition comprising chitosan, glycerol phosphate and blood or a component of said blood, and additionally ii) a pro-coagulant factor.

Alternatively, this kit comprises a pro-coagulant factor and any substance that promotes biological processes that have been identified as contributing to the formation of hyaline repair cartilage including chemotactic factors for stem cells or macrophages, bone remodelling factors, angiogenic factors, or blood or a component of said blood.

Alternatively, this kit further comprises autologous or non-autologous cells, polymer solution, blood or additional bioactive factors.

In another embodiment, the kit further comprises instructions to administer the pro-coagulant factor prior to administering the polymer composition.

In another embodiment, the kit further comprises instructions to administer the pro-coagulant factor prior to, with a delay of up to 2 minutes, prior administering the polymer composition. In another embodiment, the kit further comprises instructions to administer the pro-coagulant factor simultaneous to administering the polymer composition.

In another embodiment, the kit further comprises instructions to administer the pro-coagulant factor after administering the polymer composition.

In another embodiment, the kit further comprises instructions to administer the pro-coagulant factor with a delay of up to 2 minutes, after administering the polymer composition.

In another embodiment of this invention, there is provided the use of a pro-coagulant factor and of a polymer composition comprising a biocompatible polymer and blood or a component thereof, for repairing and/or regenerating a tissue of a patient. When the polymer composition is in contact with the pro-coagulant factor, it is converted into a non-liquid state such that the polymer composition when placed at the site in need of repair will adhere to the site in need of repair to effect repair of the tissue and/or regeneration.

In another embodiment of this invention, there is provided the use of a pro-coagulant factor and of a polymer composition comprising a biocompatible polymer and blood or a component thereof, in the manufacture of a medicament for repairing and/or regenerating a tissue of a patient. When the polymer composition is in contact with the pro-coagulant factor, it is converted into a non-liquid state such that the polymer composition when placed at the site in need of repair will adhere to the site in need of repair to effect repair of the tissue and/or regeneration.

In another embodiment of this invention, there is provided the use of a pro-coagulant factor and of a polymer composition comprising chitosan, glycerol phosphate and blood or a component thereof, for repairing and/or regenerating a tissue of a patient. When the polymer composition is in contact with the pro-coagulant factor, it is converted into a non-liquid state such that the polymer composition when placed at the site in need of repair will adhere to the site in need of repair to effect repair of the tissue and/or regeneration.

In another embodiment of this invention, there is provided the use of a pro-coagulant factor and of a polymer composition comprising chitosan, glycerol phosphate and blood or a component thereof, in the manufacture of a medicament for repairing and/or regenerating a tissue of a patient. When the polymer composition is in contact with the pro-coagulant factor, it is converted into a non-liquid state such that the polymer composition when placed at the site in need of repair will adhere to the site in need of repair to effect repair of the tissue and/or regeneration.

In another embodiment, the use further comprises the use of a growth factor, a macrophage chemotactic factor, a stem cell chemotactic factor, cells or additional bioactive factors.

For the purpose of the present invention the following terms are defined below.

The terms “polymer” or “polymer solution”, both interchangeable in the present application are intended to mean without limitation a polymer solution, a polymer suspension, a polymer particulate or powder, and a polymer micellar suspension.

The term “repair” when applied to cartilage and other tissues is intended to mean without limitation repair, regeneration, reconstruction, reconstitution or bulking of cartilage or tissues.

The term “blood” is intended to mean whole blood, processed blood, venous blood, arterial blood, blood from bone-marrow, umbilical cord blood and placenta blood. It may be enriched in platelets.

The term “blood component” is intended to mean erythrocytes, leukocytes, monocytes, platelets, fibrinogen, and thrombin. It may further comprise platelet rich plasma free of erythrocytes. In another embodiment, blood component is intended to mean any component of the blood retaining clotting properties.

The term “biocompatible polymer” is intended to mean a polymer that can be contacted with a tissue, without altering the tissue viability and that is tolerated or accepted by the tissue or the organism.

The term “patient” is intended to mean a human or an animal.

The term “solidification” is intended to mean the loss of the liquid state to the benefit of the solid state.

The term “clotting” is intended to mean a type of solidification involving formation of a blood clot.

The “tensile strength measures the force required to pull a material to the point where it breaks. The “clot strength” is measured with the maximum amplitude (MA) i.e. a direct function of the maximum dynamic properties of fibrin and platelet bonding via GPIIb/IIIa and represents the ultimate strength of the fibrin clot.

The term “sequentially” is intended to mean that the administration of the components is done in a sequence. It can be at the same time or at different times.

The term “simultaneously” is intended to mean at the same time but the components can be administered from distinct origins, i.e. the components do not need to be first mixed before being applied simultaneously.

The term “thermogelling” is intended to mean the characteristic of a polymer which becomes non-liquid at a certain temperature.

The following abbreviations are used: TF for Tissue Factor; IIa for thrombin; rVIIa for recombinant Factor VII activated form, TEG for Thromboelastograph, MA for Maximum Amplitude (in units of millimeters, a measure of clot tensile strength) tensile strength, CG for chitosan-GP/blood, bGP for beta-glycerolphosphate, FXIII for Factor XIII; FXIII-A for Factor XIII, FXIII-Aa for Factor XIII-Aa, FXIII-B for Factor XIII-B; WB for Whole blood, WBC for White Blood Cells, RBC for Red Blood Cell, TAT for thrombin/antithrombin complex.

When combined with blood or blood components the polymer could be in an aqueous solution or in an aqueous suspension, or in a particulate state, the essential characteristics of the polymer preparation being that 1) it is mixable with blood or selected components of blood, 2) that the resulting mixture is injectable or can be placed at or in a body site that requires tissue repair, regeneration, reconstruction or bulking 3) the polymer preparation does not prevent activation of the common coagulation pathway and 4) that the mixture has a beneficial effect on the repair, regeneration, reconstruction or bulking of tissue at the site of placement.

The following experiments demonstrate a method to rapidly solidify and retain polymer-blood mixtures in a surgically prepared articular defect. By applying clotting factors (activated thrombin, or tissue factor-phospholipids, or tissue factor-phospholipids-rVIIa) to the surgical defect surface immediately prior to depositing the liquid polymer-blood mixture onto the defect, the presence of thrombin, tissue factor, activated factor VII, or a mixture of these clotting factors on the surface of the defect significantly shortens the initiation phase, resulting in rapid in situ solidification while preserving or enhancing the clot-stabilizing properties of the polysaccharide scaffold and the bioactivity of the implant. Co-delivery of a small volume of clotting factor and whole blood or a homogenous mixture of polymer-whole blood also results in rapid in situ solidification. Other approaches involving homogenous mixture of clotting factors into the liquid implant fail to generate a rapid in situ solidifying hybrid clot and are described below.

The present invention will be more readily understood by referring to the following examples, which are given to illustrate the invention rather than to limit its scope.

### Example 1

### Evaluation of the Clotting Time and Clot Tensile Strength for GP/Blood), GP/Blood and Tissue Plasminogen Activator, Chitosan-GP/Blood, Chitosan-GP/Blood and Tissue Plasminogen Activator, Chitosan-GP/Blood and rVIIa, Chitosan-GP/Blood, rVIIa and Tissue Factor

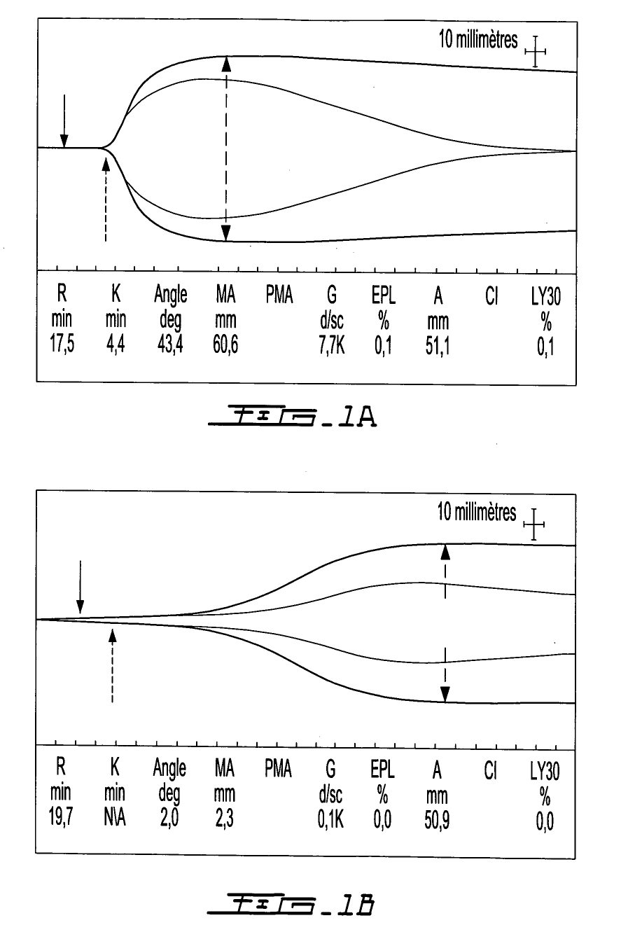

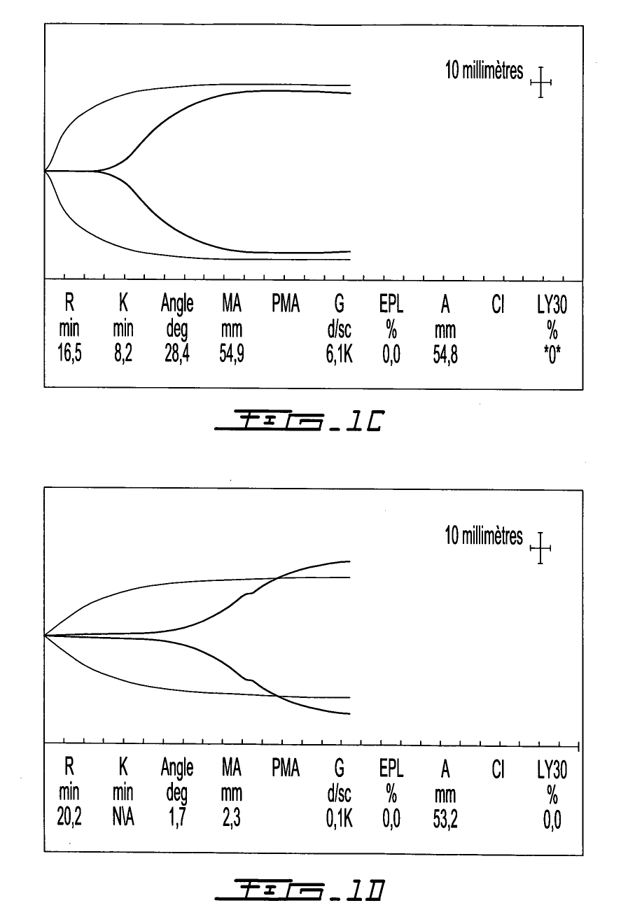

Solidification time and clot tensile strength can be evaluated using a thromboelastograph (TEG) (Bowbrick, V. A.; Mikhailidis, D. P.; and Stansby, G.: Value of thromboelastography in the assessment of platelet function. Clin Appl Thromb Hemost, 9:137-42, 2003), a type of blood rheometer. By TEG, unmodified human whole blood coagulates after a 7 to 20 minute time lapse. Samples were evaluated in a TEG set-up that allows simultaneous measurement of 8 samples, in order to evaluate the effect of clotting factors and fibrinolytic enzymes on coagulation of chitosan-GP/blood. Control samples consisted in whole blood or Glycerol Phosphate buffer (GP) without chitosan mixed with blood. Human whole blood (340 μL of freshly drawn human whole blood (FIGS. 1A,1C) was analyzed without modification, or mixed at a 3:1 ratio with near-neutral, near-isotonic solutions of either disodium beta glycerol phosphate (GP) (FIG. 1H), or chitosan-GP (FIGS. 1B,1D,1E,1F,1G). Blood, Blood-polymer (chitosan-GP/blood) or blood-buffer (GP/blood) mixtures were placed in Thromboelastograph (TEG) plastic sample cups containing 40 μL of Ringer's Lactated Saline (RLS) buffer with or without tPA (Tissue Plasminogen Activator) (FIGS. 1A-B) or clotting factors (FIGS. 1C-G) pipetted into the bottom of the cup, and analysed by TEG. The TEG trace was recorded for 3 hours. Each hash mark in FIGS. 1A, 1B, 1C, 1D, 1G, 1H is 5 minutes.

Therefore, FIG. 1 represents the evaluation of the solidification time and tensile strength for:
whole unmodified blood (black lines) and whole unmodified blood with tissue Plasminogen Activator, tPA (grey lines) (FIG. 1A),
Chitosan-GP/blood (black lines) and chitosan-GP/blood with tissue Plasminogen Activator, tPA (grey lines) (FIG. 1B),
whole unmodified blood (black lines) and whole unmodified blood with thrombin (grey lines) (IIa, FIG. 1C),
chitosan-GP/blood (black lines) and chitosan-GP/blood with IIa (grey lines, FIG. D, and FIG. 1E),
chitosan-GP/blood with rVIIa (FIG. 1G),
chitosan-GP/blood with rVIIa and Tissue Factor (TF) (FIG. 1G),
chitosan-GP/blood with and without TF (FIGS. 1E,1F,1G), and
GP/blood alone (FIG. 1H).

The results of the experiments are as follows:

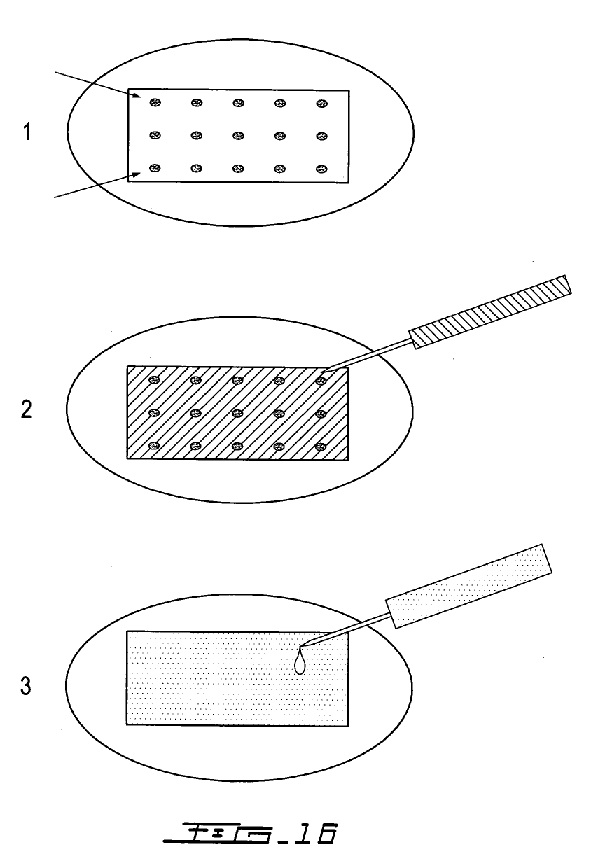

Unmodified whole blood demonstrated an initial Amplitude (A, in millimetres, mm) of 0.2 mm. Whole blood coagulated after a 17.5 minute delay (FIG. 1A) or 16.5 minute delay (FIG. 1C) and after 60 minutes acquired a tensile strength with a Maximum Amplitude (MA)=60.6 mm (FIG. 1A) or 54.9 mm (FIG. 1C). Tissue plasminogen activator (tPA), a fibrinolytic enzyme, completely dissolved the clot after 2.5 hours (FIG. 1A). Addition of thrombin (6 U/mL) to unmodified whole blood shortened the clotting time to 0.8 minutes and the time required to reach maximal tensile strength at MA=57.2 mm (FIG. 1C). GP/blood coagulated after a 13.7 minute delay (FIG. 1H).

Chitosan-GP/blood demonstrated increased viscosity at 1.3 minutes with an initial tensile strength of 2.5 to 4.5 mm (FIG. 1B). With an initial Amplitude of about 2.5 mm, coagulation occurred after 19.7 minutes and after one hour a tensile strength of MA=50.9 mm was observed. These data indicated that chitosan-GP rapidly increased whole blood viscosity, most probably due to rapid red blood cell agglutination by chitosan prior to clotting enzyme activation (Hoemann, C. D.; Sun, J.; McKee, M. D.; Chevrier, A.; Rossomacha, E.; Rivard, G. E.; Hurtig, M.; and Buschmann, M. D.: Chitosan-glycerol phosphate/blood implants elicit hyaline cartilage repair integrated with porous subchondral bone in microdrilled rabbit defects. Osteoarthritis Cartilage, 2007, 15 (1) 78-89) and/or pH-induced gelation of the polymer. The polymer-RBC thickening of the mixture at 2 minutes falsified the initial clot tensile strength measurement (MA), because the thromboelastograph only recorded the initial MA (2.5 mm) which was succeeded after 19.7 minutes by a higher amplitude which corresponded to the coagulation of blood after 19.7 minutes of coagulation. After 60 minutes chitosan-GP/blood mixtures acquired a tensile strength of Amplitude=50.9 mm (FIG. 1B). Tissue plasminogen activator (tPA) depressed chitosan-GP/blood clot tensile strength after 30 minutes (FIG. 1B) providing evidence that the clotting cascade and fibrin polymerization was involved in chitosan-GP/blood solidification. The resistance of complete lysis of the chitosan-GP/blood clot by tPA provides evidence that a RBC-chitosan scaffold resistant to the action of tPA is involved in clot stabilization.

In FIG. 1D, a clotting time of 20.2 minutes and a tensile strength of Amplitude=53.2 mm were observed for chitosan-GP/blood without IIa and a clotting time of 0.8 minutes and a tensile strength of Amplitude=41.8 mm were observed for chitosan-GP/blood with IIa.

Various concentrations of thrombin (IIa) or Tissue Factor (TF) were tested in FIG. 1E: line a contained chitosan-GP/blood (CG)+5 pM TF; line b contained CG+10.0 U IIa; line c contained CG+2.0 U IIa, line d contained CG+0.4 U IIa, line e contained CG+0.08 U IIa and line f contained CG only. In FIG. 1E, a clotting time R inferior to 2 minutes was observed in the presence of TF or IIa.

Various concentrations of TF were tested in FIG. 1F: line a contained CG+2.5 nM TF, line b contained CG+50 pM TF, line c contained CG+5 pM TF, and line d contained CG only. In FIG. 1F, a clotting time R inferior to 2 minutes was observed in the presence of TF.

Unmodified whole blood and chitosan-GP/blood solidify after a 15 to 20 minute delay in plastic recipients. Addition of thrombin, rVIIa, rVIIa with Tissue Factor, or Tissue Factor to chitosan-GP/blood shortened the time required to achieve peak clot tensile strength (FIGS. 1D, E, F & G). In FIG. 1G, a clotting time R inferior to 2 minutes was observed for TF, TF+VIIa and a clotting time R inferior to 5 minutes was observed for VIIa. In FIG. 1H, a clotting time R equal to 13.7 minutes was observed for whole blood mixed with buffer alone (beta-glycerol phosphate).

FIGS. 1A to 1D show that Chitosan-GP/whole blood mixtures solidify through activation of the common pathway. Clotting factors accelerate chitosan-GP/blood coagulation. TPA weakens chitosan-GP/blood clot strength.

Thrombin rapidly increased clot strength of whole blood and chitosan-GP/blood

FIGS. 1E to H show that Chitosan-GP/whole blood mixtures solidify via the intrinsic clotting cascade. Clotting factors accelerate chitosan-GP/blood coagulation. TPA weakens chitosan-GP/blood clot strength.

The effective concentration of clotting factor required to accelerate chitosan-GP/blood solidification in less than 2 minutes in vitro in plastic TEG sample cups was determined to be from 2 to 10 U/mL of IIa per chitosan-GP/blood (E), and from 5 to 2500 pM Tissue Factor (F). Panel G: TF and TF-rVIIa induced rapid coagulation compared to VIIa alone in chitosan-GP/blood (Panel G, CG). GP alone has little effect on coagulation (H).

### Example 2

### Quantification of Levels of Thrombin Generation Via Serum Thrombin-Antithrombin (TAT) Complex Levels

Chitosan-GP/blood clots form via thrombin generation, platelet activation and Factor XIII activation. Antithrombin is in 3-fold molar excess over pro-thrombin in plasma, and binds with very high affinity to activated thrombin. Therefore, thrombin generation can be quantified via serum thrombin-antithrombin (TAT) complex levels (Rivard, G. E.; Brummel—Ziedins, K. E.; Mann, K. G.; Fan, L.; Hofer, A.; and Cohen, E.: Evaluation of the profile of thrombin generation during the process of whole blood clotting as assessed by thrombelastography. J Thromb Haemost, 3:2039-43, 2005). To measure thrombin generation in blood-polymer and blood-buffer mixtures, clotting assays were performed in plastic tubes which do not activate the contact pathway (Factor XII). Control samples were also generated in glass vials which activate the contact pathway.

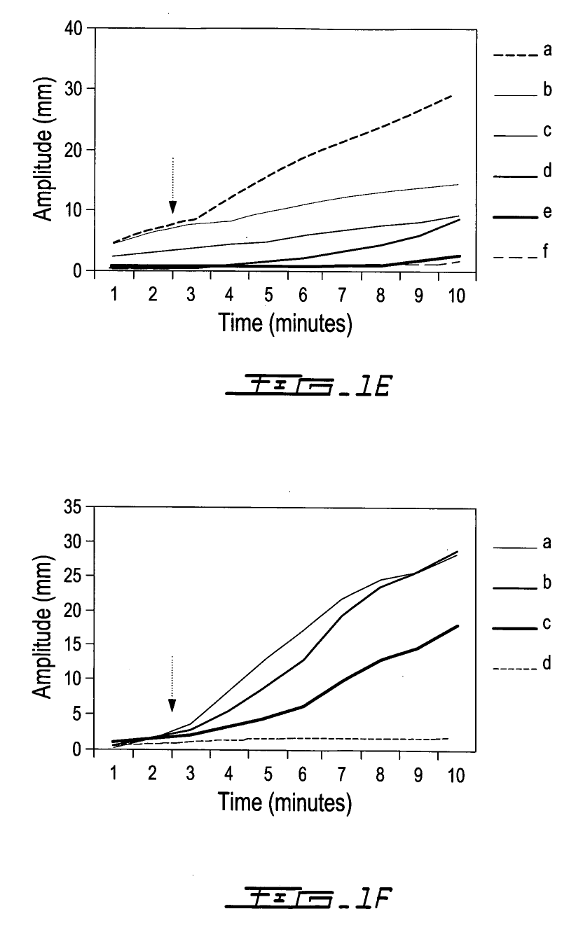

To each plastic tube, 320 μL chitosan-GP/blood (lane 1), GP/blood (lane 2), or whole blood (lane 3) with or without 40 μL buffer was pipetted and allowed to clot for 40 minutes. The clotting reaction was arrested with ice cold quenching buffer containing protease inhibitors, the serum cleared of blood cells and analyzed by ELISA for TAT, and by Western blot for the release of Platelet Factor 4 (PF4). TAT was detectable after 20 to 40 minutes of incubation at 37° C. (FIG. 2).

A significant induction of Thrombin-Antithrombin (TAT) was seen at 20 to 40 minutes post-clotting for chitosan-GP/blood (1), GP/blood (2), and whole blood (3). Sample size for each condition was as follows: (A) t=0, N=5; (B) t=20-40 minutes, N=7; and (C) glass tube samples, N=1. Samples in glass tubes experienced activation of clotting via the contact pathway which activates tenase, thrombin, and platelets.

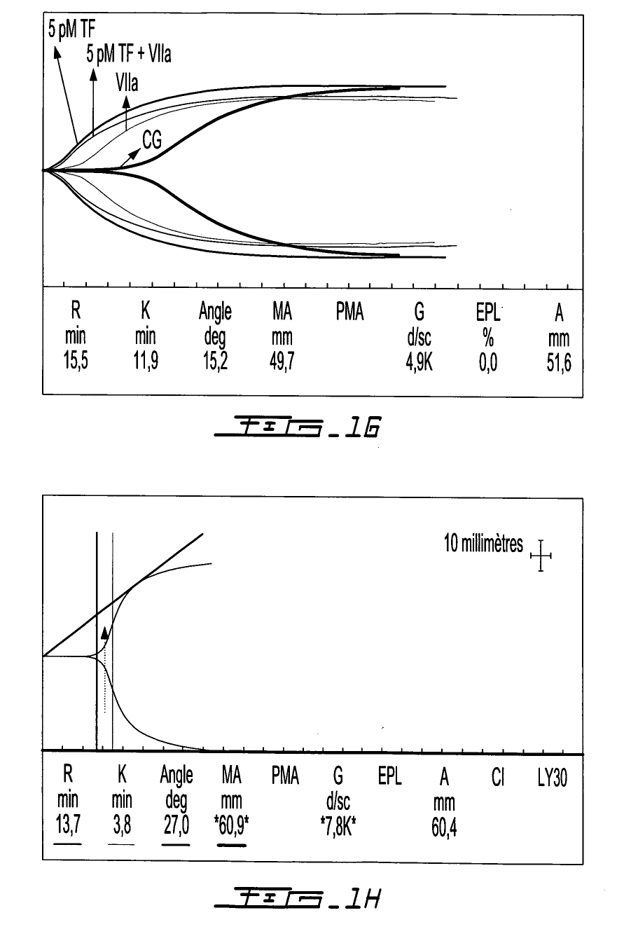

A significant correlation was observed between thrombin activation (Thrombin-Antithrombin or TAT levels) and clot tensile strength (as determined by Maximum Amplitude, MA, in mm) for both whole blood (FIG. 3A with N=26 from 3 donors; R²=0.93 and p<0.00001, with 95% confidence) and chitosan-GP/blood (FIG. 3B with N=32 from 4 donors; R²=0.93 and p<0.00001, with 95% confidence). These results suggest that chitosan-GP/blood solidification is primarily mediated by activation of the common coagulation pathway.

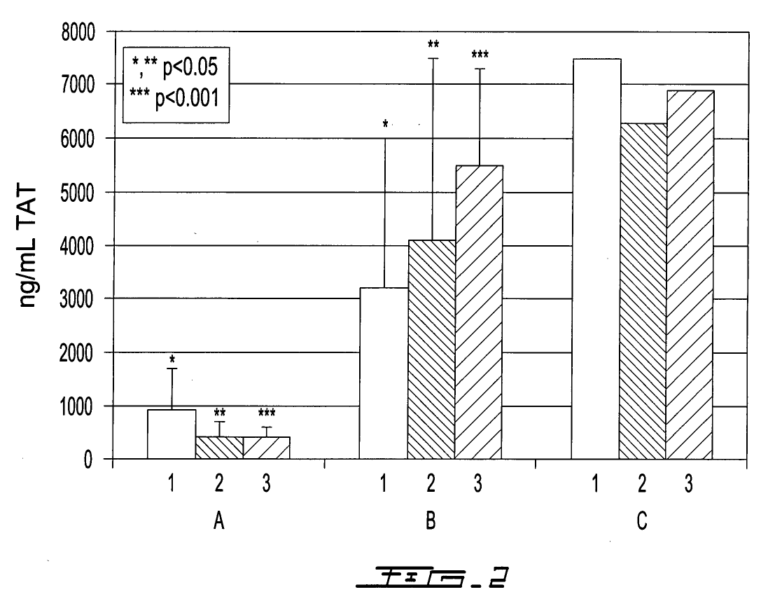

In FIG. 4, it is shown that Chitosan-GP/blood gel clot formation involves platelet activation.

In FIG. 4A, Chitosan 80 (80% DDA chitosan)-GP/Blood is used in all lanes, no buffer was added in lanes a and b and the buffer consisted in either PBS (phosphate buffered saline) in lane c, HBS (Hepes-buffered saline) in lane d, or RLS (Ringer's lactated Saline) in lane e. In FIG. 4B, No Platelet Factor 4 (PF4) was detected at t=0 in lane a, however after 40 minutes of clotting of chitosan-GP/blood (320 μL chitosan-GP/blood with or without 40 μL buffer) PF4 was detected in serum from chitosan clots (FIG. 4, lanes b to e).

In FIG. 4B, lanes a, b, g and h contained Chitosan 80 (80% DDA chitosan) GP/Blood, lanes m and n contained chitosan 95 (95% DDA chitosan) GP/blood, lanes c, d, i and j contained bGP/Blood, lanes e, f, k and l contained Whole Blood. Each sample was tested at t=0 (lanes a, c, e, g, i, k, m) and at t=40 minutes (lanes b, d, f, h, j, l, n).

Platelet Factor 4 is absent in t=0 mixtures and present at t=40 minutes at 37° C. for chitosan-GP/blood samples with and without a small volume of added buffer present (FIG. 4B).

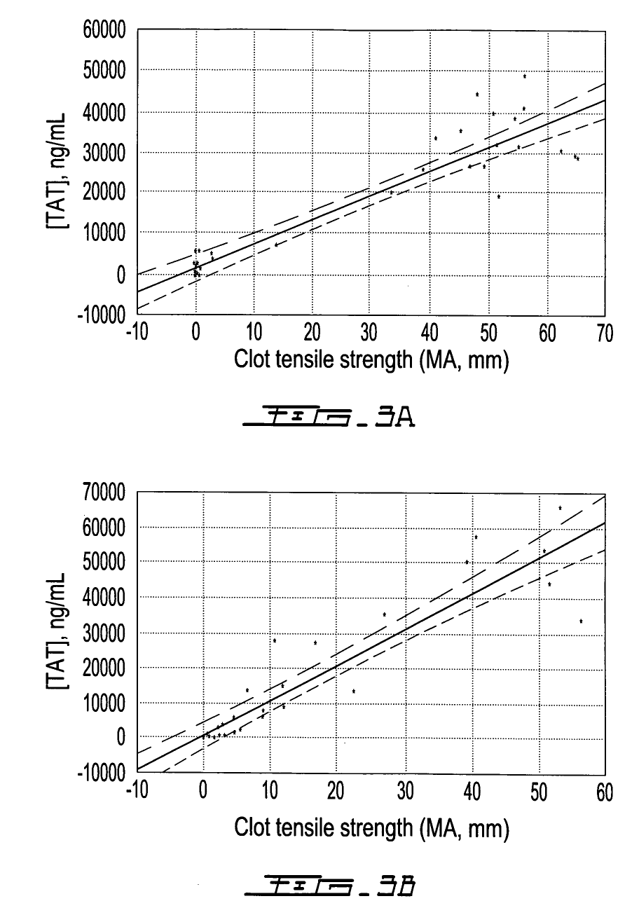

Chitosan-GP/blood gel clot formation involves factor XIII activation. In FIG. 5A, chitosan GP/Blood (Lanes a and b), bGP/Blood (Lanes c and d) and Whole Blood (Lanes e and f) samples were tested at t=0 (Lanes a, c, e) and at t=40 min (Lanes b, d, f). Activated Factor XIII (Factor XIIIa) was detected in chitosan-GP/blood, GP/blood, and whole blood after 40 minutes of coagulation (FIG. 5A) as shown by proteolytic cleavage of the 88 kDa FXIII-A subunit in a reducing SDS-PAGE Western Blot. By comparison, in FIG. 5B, on a non-reducing gel, chitosan GP/Blood (Lane a), GP/Blood (Lane b) and Whole Blood (Lane c) samples where clotting factors were added were tested. Lane c contains serum obtained after 2 minutes of incubation at 37° C. Lanes 1 to 4 contains serum collected from the samples after 20 minutes of coagulation at 37° C. Lane 1 is sample with Ringer's Lactated Saline (carrier) added. Lane 2 is sample with added VIIa. Lane 3 is sample with added VIIa and TF. Lane 4 is sample with added IIa. Factor XIII protease activation in chitosan-GP/blood mixtures occurred more rapidly in the presence of added clotting factor (FIG. 5B). These results indicate that more rapid thrombin activation leads to rapid cross-linking of the polymer-fibrin chitosan gel clot by factor XIIIa.

Solid clots were formed in glass tubes with human whole blood or chitosan-GP/blood and incubated for 20 minutes or 4 hours at 37° C. Platelet factors were detected in all serum at 20 minutes (FIG. 2E, EGF). Newly synthesized chemotactic and survival factors not present at 20 minutes were released into the serum collected from both blood clots and chitosan-GP/blood clots at 4 hours (FIG. 2E). These factors were produced by viable leukocytes trapped in the fibrin or fibrin-polymer clot. Chitosan-GP/blood clot leukocytes released elevated levels of factors specifically known to be chemotactic for macrophages and stem cells: Macrophage Chemoattractant Protein-1 (MCP-1), IL-8, Eotaxin, Macrophage Inflammatory Protein-1 alpha (MIP-1 alpha), MIP-1 beta, G-CSF (Table 1). Inflammatory chemokines and growth factors present in serum were measured by proteomic bead array immediately after clotting (20 minutes), or after 4 hours of culture of solid clots at 37° C. Chitosan-GP/blood clots and whole blood clots both released elevated levels of IL-8, MCP-1, Eotaxin, EGF, and G-CSF, but only chitosan-GP/blood clots additionally released MIP-1 beta.

TABLE 1
4 hour clot reproducible chemokine signatures (average ± SD, pg/mL)

| Chemokine | | | | | | | | | |
|---|---|---|---|---|---|---|---|---|---|
| AVE Fold- | | | | | | | | | |
| induction | CXCL8/ | | | | | | | | |
| over | IL-8 | CCL2/ | | | | | MIP-1b | GM- | |
| baseline | 1000 | MCP-1 3 | Eotaxin 3 | EGF 8 | G-CSF 3 | MIP-1a 4 | 20 | CSF 1 | IL-7 1 |
| WB clot | 3 (2) | 217 (163) | 36 (17) | 45 (12) | 76 (53) | 116 (84) | 85 (78) | 20 (17) | 41 (16) |
| t = 20 min. | | | | | | | | | |
| (N = 4) | | | | | | | | | |
| Whole | 2,674 (1476)## | 783 (464)* | 89 (42)* | 202 (92)## | 202 (154) | 278 (256) | 387 (359) | 30 (35) | 43 (4) |
| Blood | | | | | | | | | N = 5 |
| clot (N = 9) | | | | | | | | | |
| cultured | | | | | | | | | |
| 4 hr | | | | | | | | | |
| Chitosan- | 3,968 (2807)## | 696 (306)# | 105 (45)* | 107 (73)* | 255 (148)* | 417 (225)* | 1,830 (4733) | 18 (22) | 41 (15) |
| GP | | | | | | | | | N = 7 |
| clot | | | | | | | | | |
| (N = 11) | | | | | | | | | |
| cultured | | | | | | | | | |
| 4 hr | | | | | | | | | |

*p < 0.05,
#p < 0.005,
##p < 0.001 versus whole blood clot serum at 20 min post-clotting (N = 4)

Results shown in Examples 1 and 2 demonstrate that coagulation of unmodified whole blood is accelerated by the addition of clotting factors Factor VIIa, IIa, and TF-phospholipids. Unmodified whole blood already contains calcium which is required for clotting factor VII, II, X-phospholipid interaction. Introduction of activated thrombin into unmodified whole blood directly promotes platelet activation and the common pathway. Introduction of TF-phospholipids into whole blood activates the extrinsic pathway through contact between TF and low levels of circulating Factor VIIa, leading to extrinsic tenase activity and activation of the common pathway. In the presence of TF-phospholipids, addition of excess Factor VIIa will accelerate extrinsic tenase production through more effective competition of VIIa with non-activated plasma Factor VII for TF binding. Addition of Factor VIIa alone will accelerate coagulation of whole blood, partly because Factor VIIa activates platelets. Fibrin polymerization in unmodified whole blood or mixtures of unmodified whole blood mixed with other substances will be naturally accelerated by the addition of thrombin, or TF-phospholipids, or TF-phospholipids-VIIa, provided that fibrinogen, calcium, and Factor XIII are present and that further addition of other substances does not interfere with the common pathway.

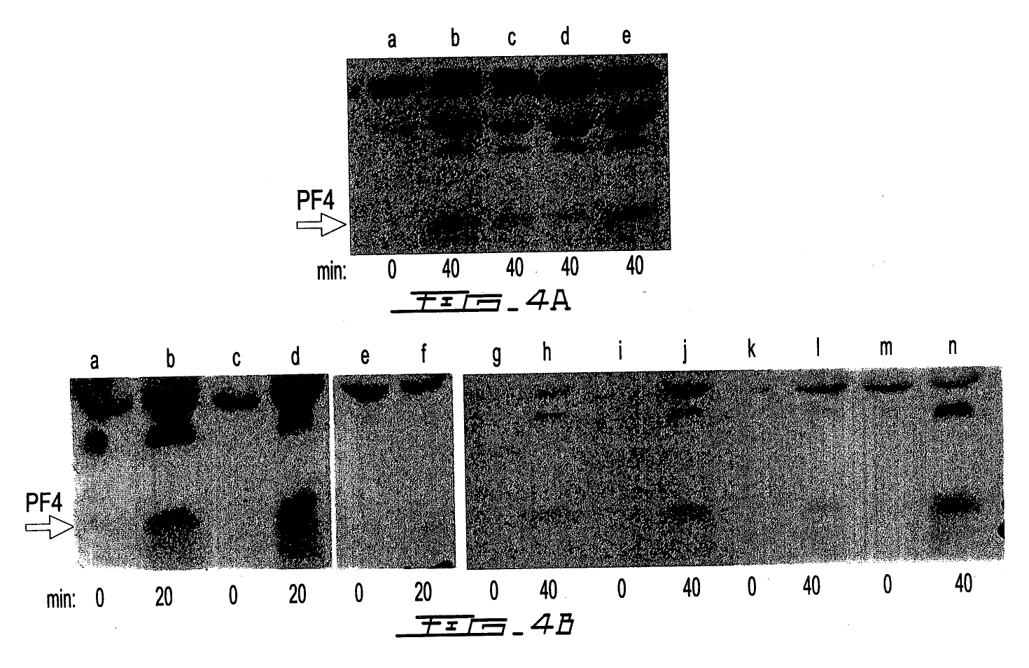

These collective results support a model demonstrating the mechanism of solidification of polymer-blood mixtures (FIG. 6), which provides a simplified schema explaining how clotting factors (Tissue Factor, Thrombin) or platelet-activating factors accelerate solidification of polymer/blood mixture. The factors accelerate polymerization of a cross-linked fibrin network around the polymer and white blood cells (WBC). Red Blood Cells are not shown. Platelets release mitogens, angiogenic factors, and chemotactic factors, while WBC release stem cell and leukocyte chemotactic factors.

### Example 3

### Comparison of In Situ Solidification

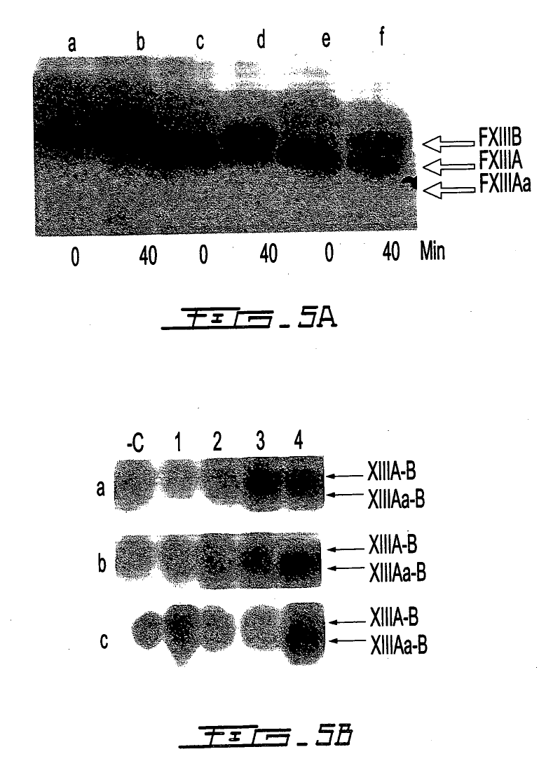

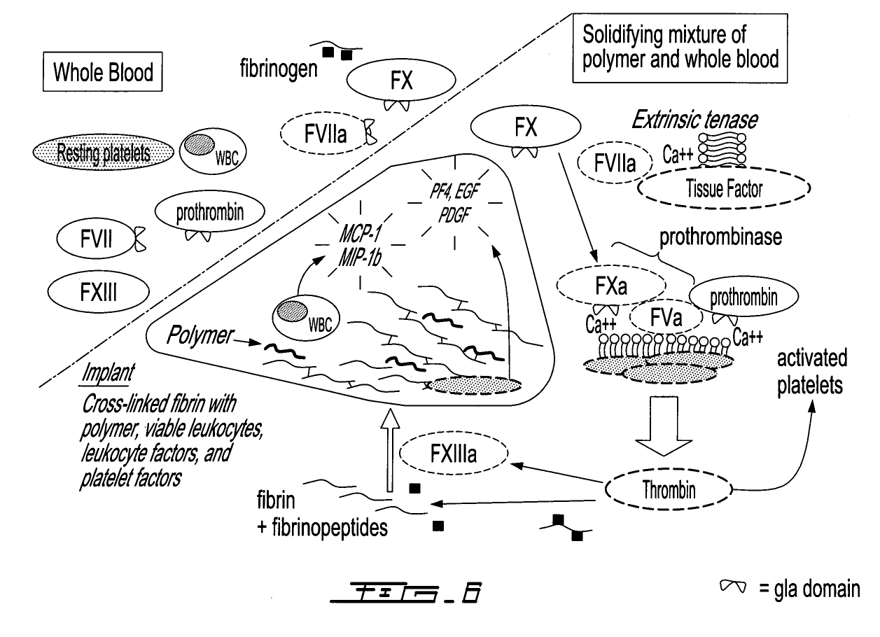

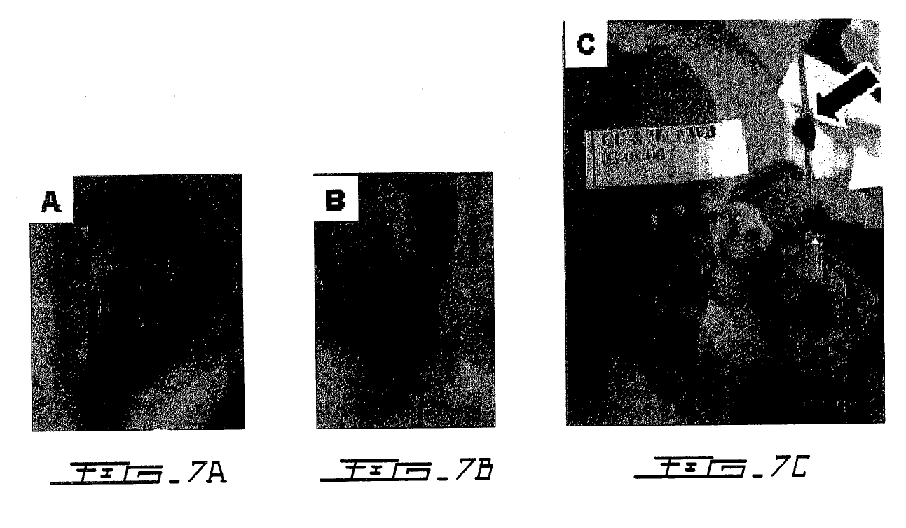

These tests used rabbit whole blood. In individual mixing vials, clotting factors TF-phospholipids ±rVIIa or thrombin were homogenously mixed into the blood or the chitosan-GP solution prior to combining the blood and chitosan-GP solution. FIG. 7 describes the generation of polymer-blood mixtures using chitosan-GP alone (FIG. 7A); chitosan-GP mixed with TF-rVIIa then whole blood (FIG. 7B) and chitosan-GP mixed with IIa then whole blood (FIG. 7C). FIG. 8 describes the use, on either plastic vials (the three samples on the right side of FIG. 8A and the samples on the left side of the FIG. 8B) or on glass vials (the four samples on the left side of the FIG. 8A and the samples on the right side of FIG. 8B), of the following samples:

1. Chitosan-GP mixed with blood (solid implant, arrow)

2. Chitosan-GP mixed with TF-rVIIa then blood

3a. Chitosan-GP mixed with TF then blood

3b. Chitosan-GP mixed with TF then blood-rVIIa

Immediately following mixture of the polymer solution with or without clotting factors and blood, the samples were then deposited on ex vivo cartilage defects (FIG. 7), or in test tubes at 37° C. (FIG. 8). As previously observed (Hoemann, C. D.; Hurtig, M.; Rossomacha, E.; Sun, J.; Chevrier, A.; Shive, M. S.; and Buschmann, M. D.: Chitosan-glycerol phosphate/blood implants improve hyaline cartilage repair in ovine microfracture defects. J Bone Joint Surg Am, 87: 2671-86, 2005), chitosan-GP/blood generated a voluminous clot in the ex vivo defect within one hour (FIG. 7A). The chitosan-GP/blood mixture without additional clotting factors (sample 1) formed a solid implant, because this mixture experiences contact activation (glass) or with time spontaneous platelet activation (plastic) which both lead to prothrombinase assembly on activated platelets. In each case tenase has unbridled access to pro-thrombin for propagation of the common pathway. Mixture of TF ±rVIIa into the blood or polymer solution prior to mixture generated semi-solid implants (FIGS. 7B, 8A & 8B, samples 2, 3a and 3b). In FIG. 7B, the chitosan-TF-rVIIa/blood implant soaked into the subchondral bone. This composition failed to form a solid implant rapidly enough to avoid soaking into the bone. Homogenous mixture of IIa directly into the polymer, blood, or polymer-blood mixture resulted in instantaneous solidification of the polymer-blood mixture in the mixing vial and syringe and could not be extruded by the needle (FIG. 7C, black arrow). Homogenous mixture of TF-phospholipids or IIa directly into the polymer prior to combining with blood followed by injection into an ex vivo defect failed to generate an in situ-solidifying, firm hybrid clot implant, that is, a solid implant. It is most probable that pre-mixture of TF-phospholipids with chitosan-GP solution led to complex formation between cationic chitosan and the negatively charged phospholipids or clotting factor gla domains, which is predicted to inhibit assembly of tenase on the surface of the phospholipids, or else which is predicted to promote the formation of “decoy” inactive complexes of chitosan-phospholipid-Factor X for example. However in the case of EXAMPLE 1, FIG. 1, addition of clotting factors TF-phospholipids ±rVIIa (extrinsic tenase) to chitosan-GP/blood accelerated solidification of the chitosan-GP/blood mixture because when chitosan was homogenously mixed with whole blood prior to coming in contact with TF-phospholipids, chitosan became opsonized by serum factors, which neutralized the positive chitosan charge thus preventing chitosan from competing with clotting factor gla-domains for binding with negatively charged phospholipids which are required for extrinsic tenase complex formation.

Chitosan-GP/blood generated a voluminous clot in the glass test tube (FIG. 8A, sample 1 & FIG. 8B, samples 1). None of the clotting factor-polymer/blood homogenous mixtures formed a voluminous clot (FIGS. 7B & 8B). Therefore, homogenous mixture of those clotting factors shown in FIGS. 8 A and B, samples 2, 3a and 3b) into the blood or polymer solution prior to mixture did not improve the practical handling of the polymer-blood mixture as an injectable in situ solidifying implant. These results indicate that the polymer mixed into whole blood must be in a form that does not inhibit assembly of the tenase with phospholipids or pro-thrombinase complex on activated platelet membranes. Mixture of rVIIa into the blood or polymer-blood solution will generate a composition that is primed for rapid clot activation when subsequently combined with Tissue Factor-phospholipids.

Application of clotting factors to a solid substrate prior to deposition of the liquid implant results in rapid in situ solidification.

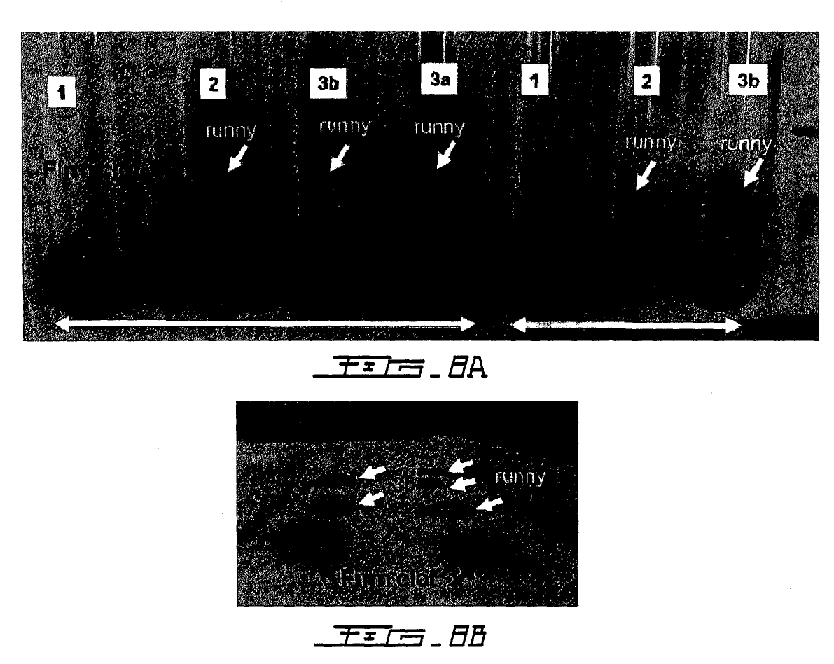

In situ solidification of whole rabbit blood. Tests were performed with rabbit whole blood as shown on FIG. 9. Spots of 1 μL to 10 μL of clotting factor in an isotonic neutral buffer were first deposited at distinct points on a glass or plastic Petri, followed by 2 to 3 drops of rabbit whole blood or chitosan-GP/blood (CG) mixtures (FIG. 9A). The clotting factors tested included thrombin, Tissue Factor, Tissue Factor+rVIIa, and rVIIa. In FIG. 9A, on plate A1, 5 μL of each clotting factor was spotted, followed by 2 drops of rabbit whole blood. Whole blood (WB) (lane 1) and WB-rVIIa (lane 3) were runny while WB-TF-rVIIa (lane 2), WB-TF (lane 4), or WB-IIa (lane 5) solidified rapidly in situ. In FIG. 9A, on plate (A2), different quantities of clotting factors were deposited: lanes 1 and 5 received 2 μL, lanes 2 and 6 received 5 μL, lanes 3 and 7 received 10 μL of clotting factor IIa. Thrombin at 10 μL (final concentration=2 U/mL) IIa from 2 distinct suppliers (Haematologic Technologies, USA, in lanes 1 to 3 and Sigma, Oakville, Canada, in lanes 5 to 7) was sufficient to induce rapid in situ clotting (less than 1 minute) of rabbit whole blood (open arrow) on a plastic Petri. Tissue Factor (2.5 nM) and phospholipids (Dade Boehring, USA) in lane 4 was also sufficient to induce rapid in situ clotting (less than 1 minute) of rabbit whole blood.

The plate was tilted at a 45° angle after a 1 minute delay to observe rapid in situ solidification. This experiment showed that rapid in situ solidification occurred with TF, TF-rVIIa and IIa but not with rVIIa alone of both rabbit whole blood (FIG. 9A) and chitosan-GP/blood mixtures (FIG. 9B).

In situ solidification of mixtures of polymer-whole blood. Freshly mixed liquid chitosan-GP/blood was dripped onto Tissue Factor-rVIIa on a plastic plate (FIG. 9B). This drip test revealed that chitosan-GP/blood applied to a small volume containing a certain amount of Tissue Factor-phospholipids-rVIIa (3 μL with a final concentration of approximately 2.5 nM tissue factor with phospholipids, 5 μg/mL rVIIa) (2^{nd} lane) was completely solid within 60 seconds (open arrow) while the blood (3^{rd} lane) or chitosan-GP/blood mixture (1^{st} lane) was still runny Therefore, pre-application of IIa, tissue factor-phospholipids, or tissue factor-phospholipids+rVIIa to the surface requiring treatment with implant can accelerate clotting in situ and improve the practical handling of the polymer-blood mixture as an injectable in situ solidifying implant.

In situ solidification of whole human blood or polymer-human blood mixtures. Tests were performed with human whole blood.

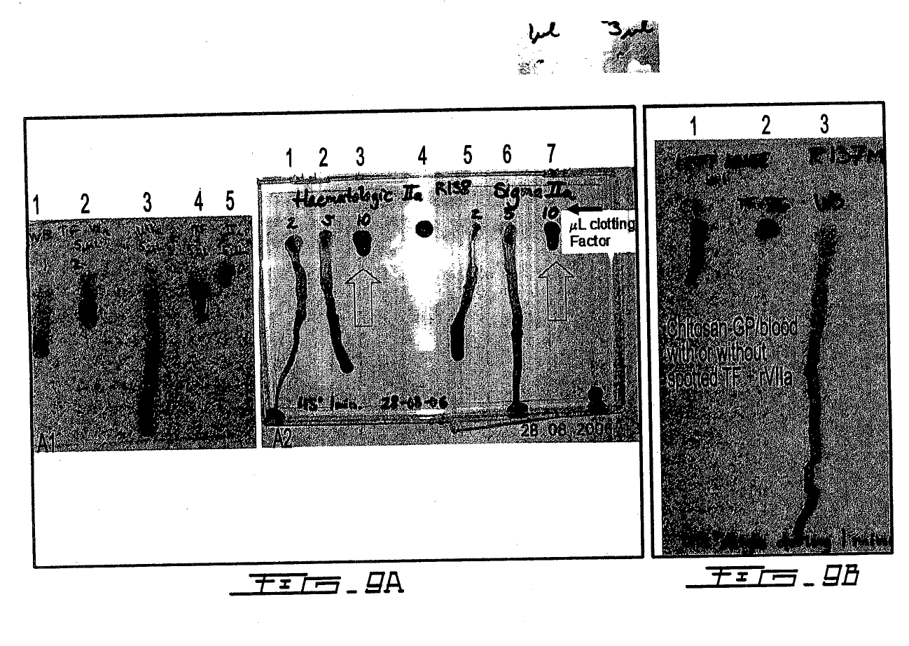

Co-application of clotting factors and liquid implant (blood or blood-polymer mixtures) to a solid substrate, such a plastic Petri results in rapid in situ solidification. The clotting factors tested included thrombin and Tissue Factor. A pipette tip with 3 μL of clotting factor was placed in contact with a second pipette tip containing 50 μL of whole blood or polymer composition mixed with blood. The solutions were simultaneously expelled from the pipette tips, forming a single drop which mingled the clotting factors and blood or polymer-blood mixture. The plate was tilted vertically after a 30 second delay to observe rapid in situ solidification. This experiment showed that rapid in situ solidification occurred with TF (FIG. 10A) and thrombin (FIG. 10B). The level of TF needed to induce rapid in situ solidification was between 250 and 2500 pM final concentration (FIG. 10A, lanes 2, 3, and 6). In FIG. 10A, the samples contained 50 μL of blood and 3 μL of tissue Factor-phospholipids (TF) in order to obtain a final concentration of 0 (lane 1), 500 (lane 2), 250 (lane 3), 25 (lane 4), 5 (lane 5) and 2500 (lane 6) pM of Tissue factor. The level of IIa needed to induce rapid in situ solidification of chitosan-GP/blood was between 2 and 10 U/mL (FIG. 10B, where the sample contained 50 μL of chitosan-GP/blood and 3 μL of IIa to obtain a concentration of approximately 2 U/mL of IIa). Therefore, simultaneous administration of blood mixed with polymer or factors, and a small volume of clotting factor can accelerate clotting in situ and improve the practical handling of the liquid mixture as an injectable in situ solidifying implant.

### Example 4

### In Vivo Studies

14 rabbits were submitted to bilateral knee surgeries using small arthrotomies to create articular cartilage defects in the trochlear groove with microdrill holes. Different implants of chitosan-GP/blood with or without an additional clotting factor were applied. The arthrotomy site was closed immediately after implant solidification was observed. Defects were allowed to heal for 3 weeks (N=11) or 8 weeks (N=3).

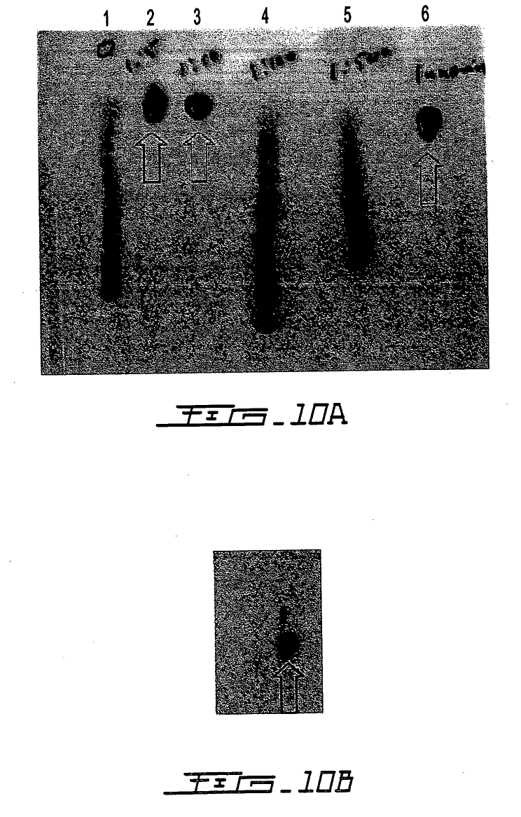

FIG. 11A shows the drilled defect (panel 1) and the defect treated with 3 μL, IIa and one drop (25 μL) chitosan-GP/blood which becomes solid in less than 90 seconds (panel 2). FIG. 11B shows the control defect after microdrill only, but untreated or treated with clotting factor and no implant.

The conditions of the studies are shown in Table 2:

TABLE 2
In vivo studies with rabbits

| | | Chitosan | | | | | |
|---|---|---|---|---|---|---|---|
| | | dosage | Clotting | | No. | | |
| Treatment | Contralateral | levels | factor | | Animals | | Repair |
| Group | Control | (mg/kg) | dosage | Dose volumes | M | F | Period |
| 1. chitosan-GP + | chitosan- | 0.012* | 200 ng | 2 × 5.6 microliters | 2 | 2 | 3 |
| blood/rVIIa | GP/blood | | rVIIa | chitosan-GP | | | weeks |
| | | | | 2 × 22.5 microliters | | | |
| | | | | blood | | | |
| | | | | 0.4 microliters | | | |
| | | | | rVIIa per implant | | | |
| 2. TF**-rVIIa + | chitosan- | 0.012* | 200 ng | 2 × 5.6 microliters | 1 | 2 | 3 |
| chitosan-GP | GP/blood | | rVIIa | chitosan-GP | | | weeks |
| | | | 5 pM TF | 2 × 22.5 microliters | | | |
| | | | | blood | | | |
| | | | | 0.25 microliters | | | |
| | | | | rVIIa and | | | |
| | | | | 5 microliters TF | | | |
| | | | | per 1 defect | | | |
| 3. IIa paint + | no implant (drilling | 0.006 | 0.05 to | 5.6 microliters | 1 | 3 | 3 |
| chitosan- | only) | | 0.45 U IIa | chitosan-GP | | | weeks |
| GP/blood | | | | 22.5 microliters | | | |
| | | | | blood | | | |
| | | | | 3 microliters IIa | | | |
| | | | | at 2 to 10 U/mL | | | |
| 4. IIa paint + | no implant (drilling | 0.006 | 0.15 U IIa | 5.6 microliters | 1 | 2 | 8 |
| chitosan- | only) | | | chitosan-GP | | | weeks |
| GP/blood | | | | 22.5 microliters | | | |
| | | | | blood | | | |
| | | | | 3 microliters IIa | | | |
| | | | | at 2 to 10 U/mL | | | |

2 chitosan-GP/blood implants per rabbit;
**TF includes phospholipids

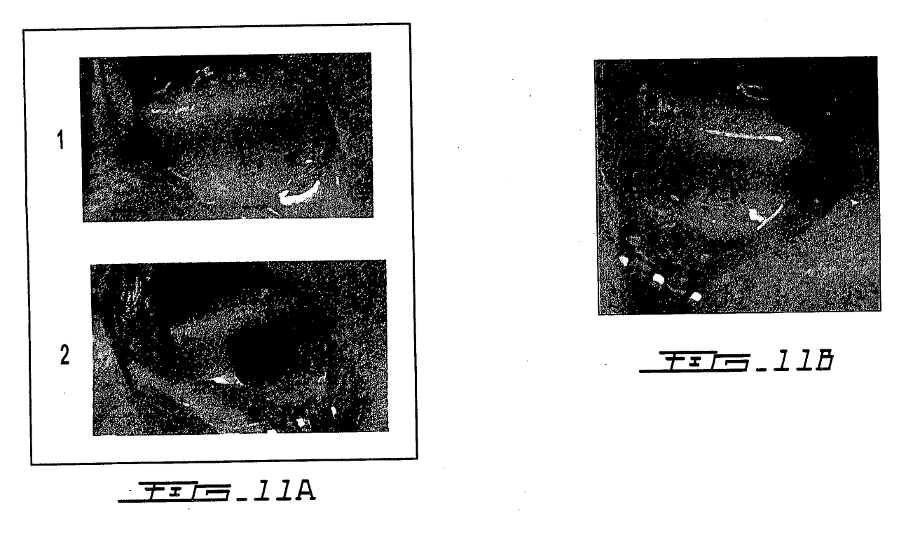

Chitosan-GP/blood mixtures alone (no clotting factor) solidified within an average time of 4 to 6 minutes (FIG. 12, lane 1, with N=7). Chitosan-GP/blood mixtures further combined homogenously with human rVIIa (Table 2, Group 1), solidified in situ after a 3 to 4 minute delay (FIG. 12, lane 2, with N=4). Since defects loaded with chitosan-GP/blood solidified with similar kinetics as chitosan mixed with whole blood/Factor VIIa, these data demonstrated that mixture of rVIIa alone into the liquid implant is insufficient to improve practical in situ solidification of chitosan-GP/blood. These results show that in order for rVIIa to accelerate clotting in situ, that a certain level of Tissue Factor (with phospholipids) needs to be present on the surface of the surgically prepared defect, or co-delivered with the liquid preparation.

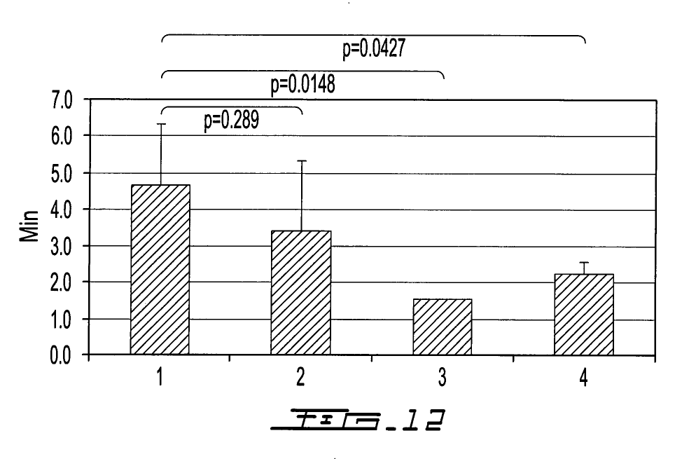

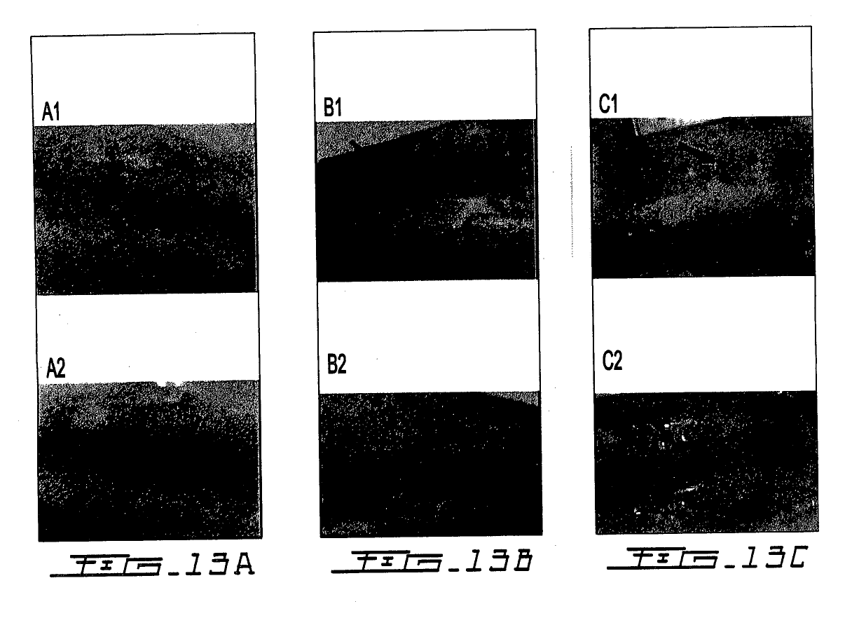

In other rabbits, the animal surgeon deposited 3 μL of clotting factor (either a mixture of TF-rVIIa, or activated thrombin in isotonic and neutral pH disodium glycerol phosphate buffer) on a microdrilled rabbit trochlear defect prior to depositing one drop of chitosan-GP/blood (Table 2, Groups 2 and 3, respectively). A delay of 5 to 10 seconds occurred between painting the clotting factor on the defect and depositing the fresh chitosan-GP/blood mixture. These implants solidified in 90 seconds or less (FIG. 12, lane 3, with N=3, for TF-rVIIa paint with chitosan-GP/blood) or in about 2 minutes (FIG. 12, lane 4, with N=4, for IIa paint, with chitosan-GP/blood). Implant deposited on defects treated with TF-rVIIa or IIa solidified significantly faster than implants deposited on untreated surgical defects in the contralateral knee (FIG. 12, p<0.05). After 3 weeks of repair, the repair tissues were examined (FIG. 13). FIG. 13A shows Rabbit R130F, with the combination of TF-rVIIa with chitosan-GP/blood treated repair, showing angionesis with bone remodeling in A-1 and the contralateral defect chitosan-GP/blood treated repair showing angiogenesis with bone remodeling in A-2. FIG. 13B shows Rabbit R134M, with a 0.1 U IIa and chitosan-GP/blood treated repair at 3 weeks showing angiogenis and remodeling in B-1 and a contralateral defect Drilling only showing fibrous tissue and no evidence of bone remodeling in B-2. FIG. 13C shows Rabbit R138F, with a 0.05 U IIa and chitosan-GP/blood treated repair showing angiogenesis, and bone remodeling in C-1, and the contralateral defect drilling showing only fibrous tissue, but no evidence of bone remodeling in C-2.

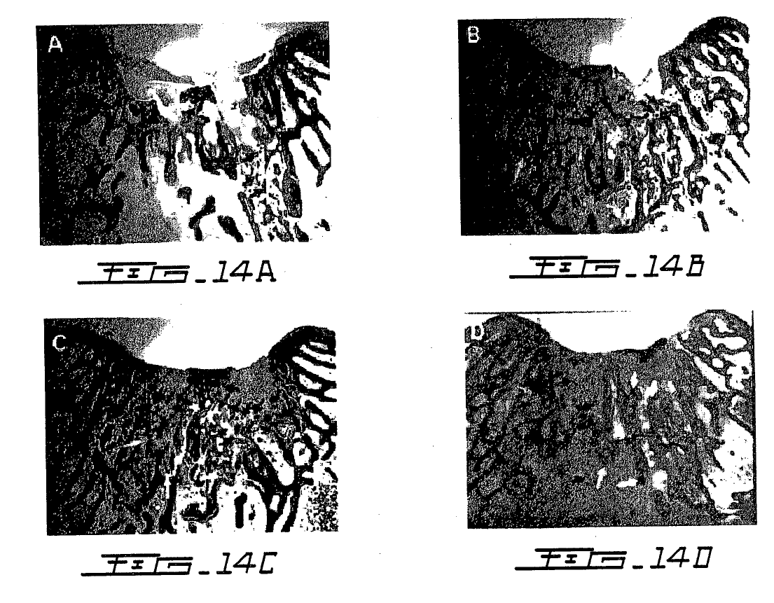

After 3 weeks of repair, both chitosan-GP/blood and chitosan-GP/blood with clotting factor showed a distinct repair response compared to drilling alone (FIGS. 13 and 14). The repair tissue resulting from defects treated with both clotting factor and chitosan-GP/blood showed an equivalent favorable biological response in terms of angiogenesis and bone remodeling that was comparable to chitosan-GP/blood alone after 3 weeks of repair (FIGS. 13 and 14, and hyaline cartilage repair after 8 weeks of repair (FIG. 14). FIG. 14 shows the histology of drilled defects without implant after 3 weeks of repair (A) versus drilled defects treated with chitosan-GP/blood alone (B) or drilled defects painted with TF-rVIIa (C) or thrombin (D) followed by chitosan-GP/blood after 3 (B & C) or 2 weeks (D) of repair. Red arrows point to blood vessels and yellow arrows to bone remodeling.

FIG. 14A shows the trochlear osteochondral repair tissue of Rabbit R133L which received no implant. FIG. 14B shows the repair tissue of Rabbit R130L which received a chitosan-GP/blood implant. FIG. 14C shows the tissue of Rabbit R130R, which received a chitosan-GP/blood implant with TF-rVIIa. FIG. 14D shows the tissue of Rabbit R176R, which received a chitosan-GP/blood implant with IIa. Stimulation of subchondral angiogenesis and bone remodeling provide evidence that the clotting factor and implant as well as shortened open arthrotomy time (˜4 minutes less) have generated a favorable repair response.

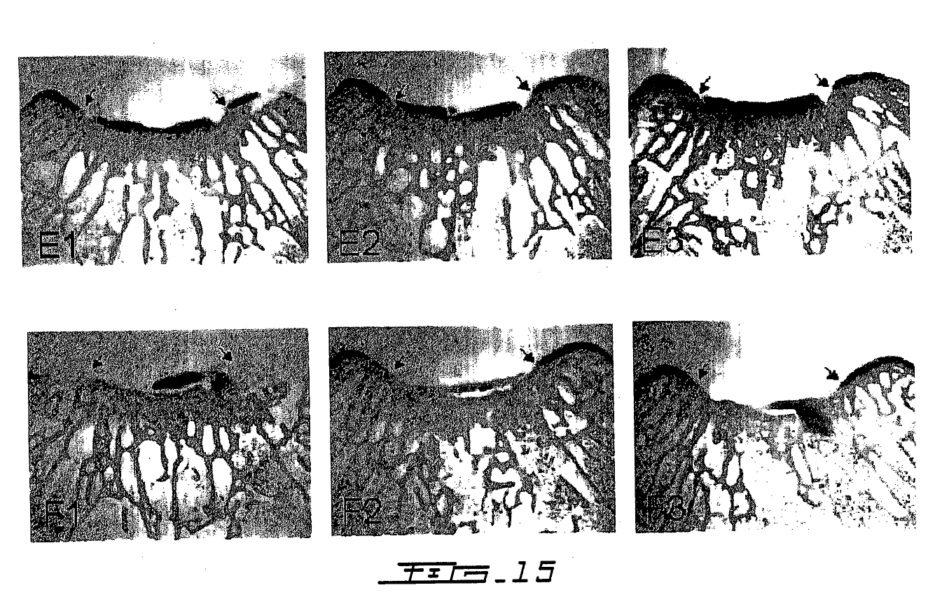

FIG. 15 shows the SafraninO-Fast green stained histology sections of bilateral repair tissue after 8 weeks of repair. One defect was drilled and treated with IIa and chitosan-GP/blood with rapid in situ solidification (panels E1-E3). The contralateral defect was drilled and treated with IIa (panels F1-F3). Implant and clotting factor showed superior repair (E) compared to defects treated with clotting factor alone (F). SafraninO (red stain) demonstrates hyaline cartilage repair tissue formed in the defect (between arrows).

To summarize the findings of Example 4:

Mixture of rVIIa alone into liquid chitosan-GP/blood mixtures does not accelerate in situ solidification in vivo within an efficient time frame.

Pre-application of clotting factor thrombin or activated tissue factor pathway (tissue factor-phospholipids-rVIIa) to a prepared surgical articular cartilage defects in vivo generates a rapid in situ solidifying implant that has equivalent biological activity (angiogenesis and bone remodelling leading to hyaline cartilage repair) to chitosan-GP/blood implants without clotting factor.

Treatment of marrow stimulated defects with clotting factor and chitosan-GP/blood implant promotes angiogenesis and bone remodelling during intermediate phase repair, and hyaline cartilage repair at an 8-week end-point.

These data demonstrate that rapid in situ solidification can be achieved using a delivery device which deposits a certain amount of activated clotting factor (Tissue Factor, Tissue Factor+rVIIa, or activated thrombin) onto the defect simultaneous with or immediately before the chitosan-GP/blood mixture is dripped onto the surface by arthroscopic delivery (i.e, 0.05 U to 0.1 U IIa painted on the defect surface per drop of implant, or 2 to 10 U thrombin per mL chitosan-GP/blood mixture).

FIGS. 16 to 18 show different procedures for realizing the invention of the present invention.

In FIG. 16, the following steps are followed:
1. The defect is confined, unconfined, horizontally placed, curved, or at a slight angle displaced from horizontal. The defect is debrided into or just below the calcified cartilage layer.
   Marrow stimulation (drill or microfracture) is performed.
2. Clotting factor(s) are applied with a wand.
3. The liquid hybrid blood mixture is immediately applied. The implant becomes solid in situ in less than 2 minutes.

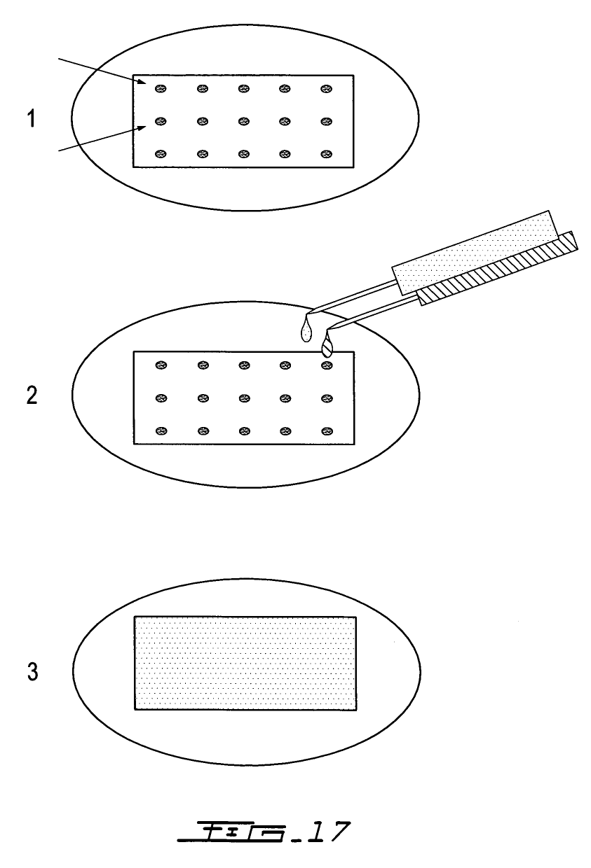

In FIG. 17, the following steps are followed:
1. The defect is confined, unconfined, horizontally placed, curved, or at a slight angle displaced from horizontal. The defect is debrided into or just below calcified cartilage layer.
   Marrow stimulation (drill or microfracture) is performed.
2. The hybrid liquid polymer-blood mixture and the clotting factor are co-applied with a co-injection of both components.
3. The implant becomes solid in situ in less than 2 minutes.

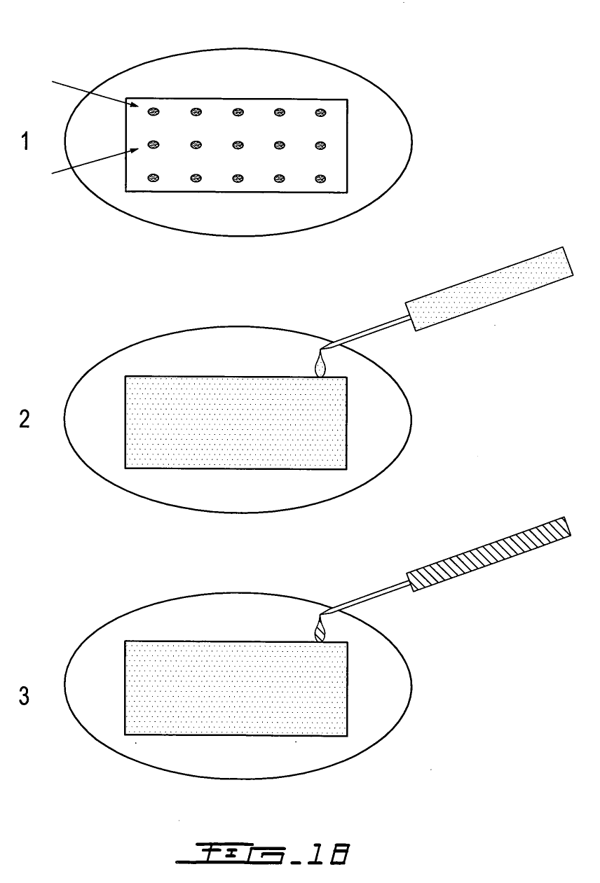

In FIG. 18, the following steps are followed:
1. The defect is confined, unconfined, horizontally placed, curved, or at a slight angle displaced from horizontal. The defect is debrided into or just below calcified cartilage layer.
   Marrow stimulation (drill or microfracture) is performed.
2. The hybrid blood mixture and the clotting factor are co-applied by first injecting the liquid polymer-blood mixture and applying the clotting factor by dripping or spraying.
   3. The implant becomes solid in situ in less than 2 minutes.

Examples of composition of the invention are the following:

In one composition, 1 cc of the hybrid polymer blood mixture is co-injected with a thrombin solution of 120 μL at 2 to 10 U/mL final concentration.

In one composition, 1 cc of the hybrid polymer blood mixture is co-injected with a Tissue Factor solution of 120 μL at 250-2500 pM final concentration. The Tissue Factor includes a mixture of Tissue Factor protein or ectodomain with phospholipids.

In one composition, 1 cc of the hybrid polymer blood mixture is co-injected with a Tissue Factor/rVIIa solution of 120 μL at 250-2500 pM TF/0.5-50 μg/mL rVIIa final concentration. The Tissue Factor includes a mixture of Tissue Factor protein or ectodomain with phospholipids.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All references cited herein are being hereby incorporated by reference.

1. A method for repair and/or regeneration of a tissue of a patient comprising administering into said tissue in need of repair and/or regeneration:
i. a pro-coagulant factor; and
ii. a polymer composition comprising:
a biocompatible polymer; and
blood or a blood component thereof,
wherein the polymer composition in contact with the pro-coagulant factor is converted into a non-liquid state such that the polymer composition when placed at the site in need of repair will adhere to the site in need of repair to effect repair and/or regeneration of the tissue.

2. The method of claim 1, wherein the biocompatible polymer is selected from the group consisting of a polysaccharide, a protein, and a polyamino acid.

3. The method of claim 2, wherein the polysaccharide is selected from the group consisting of chitosan, chitin, hyaluronan, glycosaminoglycan, chondroitin sulfate, keratan sulfate, dermatan sulfate, heparin, cellulose, and heparin sulfate.

4. The method of claim 2, wherein the polysaccharide is chitosan.

5.-6. (canceled)

7. The method of claim 1, wherein the blood or blood component thereof is autologous to the patient.

8. (canceled)

9. The method of claim 1, wherein the blood component comprises platelet rich plasma.

10. (canceled)

11. The method of claim 1, wherein the polymer composition further comprises a growth factor or a chemotactic factor.

12.-16. (canceled)

17. The method of claim 1, wherein the pro-coagulant factor is selected from the group consisting of a clotting factor, a platelet-activating factor, collagen, ellagic acid, epinephrine and adenosine diphosphate.

18.-23. (canceled)

24. A method for repair and/or regeneration of a tissue of a patient comprising administering into said tissue in need of repair and/or regeneration:
i. a pro-coagulant factor; and
ii. a polymer composition comprising:
chitosan;
glycerol phosphate; and
blood or blood component thereof,
wherein the polymer composition is converted into a non-liquid state, such that said polymer composition once converted into a non-liquid state adheres to the site in need of repair and/or regeneration when placed thereon to effect repair and/or regeneration of the tissue.

25. (canceled)

26. The method of claim 24, wherein the blood or component thereof is autologous to the patient.

27. (canceled)

28. The method of claim 24, wherein the blood component comprises platelet rich plasma.

29. (canceled)

30. The method of claim 24, wherein the polymer composition further comprises a growth factor or a chemotactic factor.

31.-34. (canceled)

35. The method of claim 24, wherein the pro-coagulant factor is selected from the group consisting of a clotting factor, a platelet-activating factor, collagen, ellagic acid, epinephrine and adenosine diphosphate.

36.-37. (canceled)

38. The method of claim 24, wherein administering of the pro-coagulant factor to the tissue is done either simultaneously, immediately prior to, or with a delay of up to 2 minutes, before administering the polymer composition, or immediately after, or with a delay of up to 2 minutes, administering the polymer composition.

39.-41. (canceled)

42. A kit for repair and/or regeneration of a tissue of a patient, said kit comprising i) a pro-coagulant factor and ii) a polymer composition comprising a biocompatible polymer.

43. (canceled)

44. The kit of claim 42, further comprising blood or a blood component thereof.

45. The kit of claim 42, further comprising cells or additional bioactive factors.

46. The kit of claim 42, wherein the biocompatible polymer is selected from the group consisting of a polysaccharide, a protein, and a polyamino acid.

47. The kit of claim 46, wherein the polysaccharide is selected from the group consisting of chitosan, chitin, hyaluronan, glycosaminoglycan, chondroitin sulfate, keratan sulfate, dermatan sulfate, heparin, cellulose, and heparin sulfate.

48. The kit of claim 46, wherein the polysaccharide is chitosan.

49.-50. (canceled)

51. The kit of claim 44, wherein the blood or component thereof is autologous to the patient.

52. (canceled)

53. The kit of claim 44, wherein the blood component comprises platelet rich plasma.

54. (canceled)

55. The kit of claim 42, further comprising a growth factor or a chemotactic factor.

56. (canceled)

57. The kit of claim 42, wherein the pro-coagulant factor is selected from the group consisting of a clotting factor, a platelet-activating factor, collagen, ellagic acid, epinephrine and adenosine diphosphate.

58.-62. (canceled)

63. A kit for repair and/or regeneration in a tissue of a patient comprising i) a pro-coagulant factor and ii) a polymer composition comprising chitosan and glycerol phosphate.

64. The kit of claim 63, further comprising blood or a component of said blood.

65.-67. (canceled)

68. The kit of claim 64, wherein the blood component comprises platelet rich plasma.

69. The kit of claim 63, further comprising a growth factor or a chemotactic factor.

70. (canceled)

71. The kit of claim 63, wherein the pro-coagulant factor is selected from the group consisting of a clotting factor, a platelet-activating factor, collagen, ellagic acid, epinephrine and adenosine diphosphate.

72.-120. (canceled)
